(19) United States
(12) Patent Application Publication
(10) Pub. No.: US 2025/0029683 A1
(43) Pub. Date: Jan. 23, 2025

(54) INFORMATION PROCESSING METHOD, INFORMATION PROCESSING SYSTEM, AND NON-TRANSITORY COMPUTER-READABLE RECORDING MEDIUM

(71) Applicant: Panasonic Intellectual Property Management Co., Ltd., Osaka (JP)

(72) Inventors: KOKI UENO, Osaka (JP); KENSUKE WAKASUGI, Tokyo (JP); MASATO SHIMABAYASHI, Nara (JP); AKIHIRO SAKAI, Nara (JP)

(21) Appl. No.: 18/906,211

(22) Filed: Oct. 4, 2024

Related U.S. Application Data
(63) Continuation of application No. PCT/JP2023/014134, filed on Apr. 5, 2023.

(30) Foreign Application Priority Data
Apr. 21, 2022 (JP) 2022-069946

(51) Int. Cl. G16C 20/20; G01N 23/2055; G16C 20/40

(52) U.S. Cl. CPC G16C 20/20; G01N 23/2055; G16C 20/40

(57) ABSTRACT

An information processing method is an information processing method to be executed by a computer. The information processing method includes acquiring experimental spectrum information indicating an experimental spectrum obtained by actually measuring a material to be searched for; acquiring material information regarding a composition of the material; generating, based on the material information, pieces of candidate structure information regarding candidate structures that are candidates for a crystal structure of the material, and acquiring computational spectrum information indicating computational spectra each corresponding to a corresponding one of the candidate structures; generating structure information regarding the crystal structure, based on a correlation between the experimental spectrum information and the computational spectrum information; and outputting the generated structure information.

### BACKGROUND

### 1. Technical Field

The present disclosure relates to an information processing method and the like for identifying a crystal structure.

### 2. Description of the Related Art

The physical properties of a material are mainly determined by the crystal structure thereof. Thus, a technique for identifying the crystal structure of an unknown material is demanded. For example, in MCCUSKER, L. B., et al., Rietveld refinement guidelines, Journal of Applied Crystallography, 1999, 32.1:36-50 (hereinafter referred to as NPL 1), a technique for identifying a crystal structure by the Rietveld method is disclosed.

For example, in OGANOV, Artem R.; GLASS, Colin W., Crystal structure prediction using ab initio evolutionary techniques: Principles and applications, The Journal of chemical physics, 2006, 124.24:244704 (hereinafter referred to NPL 2), a crystal structure identification technique using a density functional theory is disclosed.

For example, Japanese Unexamined Patent Application Publication No. 2021-149449 (hereinafter referred to PTL 1) discloses a method of searching for a material having predetermined characteristics by using spectrum information.

### SUMMARY

One non-limiting and exemplary embodiment provides an information processing method and the like that facilitate efficient and accurate identification of an unknown crystal structure.

In one general aspect, the techniques disclosed here feature an information processing method to be executed by a computer, the information processing method including acquiring experimental spectrum information indicating an experimental spectrum obtained by actually measuring a material to be searched for; acquiring material information regarding a composition of the material; generating, based on the material information, pieces of candidate structure information regarding candidate structures that are candidates for a crystal structure of the material, and acquiring computational spectrum information indicating computational spectra each corresponding to a corresponding one of the candidate structures; generating structure information regarding the crystal structure, based on a correlation between the experimental spectrum information and the computational spectrum information; and outputting the generated structure information.

According to an aspect of the present disclosure, it is easy to efficiently and accurately identify an unknown crystal structure.

It should be noted that general or specific embodiments may be implemented as a system, a method, an integrated circuit, a computer program, a storage medium, or any selective combination thereof.

Additional benefits and advantages of the disclosed embodiments will become apparent from the specification and drawings. The benefits and/or advantages may be individually obtained by the various embodiments and features of the specification and drawings, which need not all be provided in order to obtain one or more of such benefits and/or advantages.

### BRIEF DESCRIPTION OF THE DRAWINGS

FIG. 1 is a block diagram illustrating an overall configuration including an information processing system according to Embodiment 1;

FIG. 2 is a flowchart illustrating an overview of the operation of the information processing system according to Embodiment 1;

FIG. 3 is a diagram illustrating an example of a list of pieces of candidate structure information;

FIGS. 4A and 4B are explanatory diagrams of a method of generating candidate structure information;

FIGS. 5A and 5B are diagrams illustrating a specific example of the candidate structure information;

FIGS. 6A and 6B are diagrams illustrating another specific example of the candidate structure information;

FIG. 7 is a flowchart illustrating an example of computational spectrum calculation;

FIGS. 8A and 8B are diagrams illustrating an example of a discrete spectrum and a continuous spectrum;

FIGS. 9A and 9B are explanatory diagrams of the similarity between an experimental spectrum and a computational spectrum;

FIG. 10 is an explanatory diagram of structural optimization by the information processing system according to Embodiment 1;

FIG. 11 is a flowchart illustrating an example of an optimization step by the information processing system according to Embodiment 1;

FIG. 12 is a block diagram illustrating an overall configuration including an information processing system according to Embodiment 2;

FIG. 13 is a flowchart illustrating an overview of the operation of the information processing system according to Embodiment 2;

FIG. 14 is a flowchart illustrating an example of energy calculation;

FIG. 15 is a diagram illustrating an example of a list of energies of candidate structures;

FIG. 16 is a flowchart illustrating an example of an optimization step by the information processing system according to Embodiment 2;

FIGS. 17A, 17B, and 17C are explanatory diagrams of structural optimization by the information processing system according to Embodiment 2;

FIG. 18 is a diagram illustrating an image displayed on a display unit in the information processing system according to Embodiment 2;

FIG. 19 is a flowchart illustrating an overview of the operation of an information processing system according to Comparative Example 1;

FIG. 20 is a flowchart illustrating an overview of the operation of an information processing system according to Comparative Example 2;

FIG. 21 is a histogram illustrating similarities between an experimental spectrum and computational spectra of a structure group structurally optimized in Example 1;

FIG. 22 is a histogram illustrating similarities between an experimental spectrum and computational spectra of a structure group structurally optimized in Example 2;

FIG. 23 is a histogram illustrating similarities between an experimental spectrum and computational spectra of a structure group structurally optimized in Comparative Example 1;

FIG. 24 is a histogram illustrating similarities between an experimental spectrum and computational spectra of a structure group structurally optimized in Comparative Example 2;

FIG. 25 is a diagram illustrating a comparison between an experimental spectrum and computational spectra of structures having the highest similarity in Examples 1 and 2 and Comparative Examples 1 and 2;

FIG. 26 is a diagram illustrating an example of one or more pieces of structure information; and

FIG. 27 is a diagram illustrating an example of information included in an “output” area in FIG. 18.

### DETAILED DESCRIPTIONS

### Underlying Knowledge Forming Basis of the Present Disclosure

Conventionally, identification of a crystal structure has been carried out by analyzing an experimentally obtained X-ray diffraction pattern of an unknown material. NPL 1 discloses a technique for identifying a crystal structure by the Rietveld method. In the conventional identification of a crystal structure by the Rietveld method, however, it is necessary to start the identification of the crystal structure from one candidate structure considered to be closest to the actual crystal structure. Further, the parameters of the candidate structure need to have values close to the parameters of the actual crystal structure, and are difficult to apply to an unknown crystal structure.

In addition, with the development of simulation technology in recent years, for example, a method of identifying a thermodynamically stable crystal structure by computing the energies of a large number of crystal structures using a density functional theory has been used. That is, a crystal structure considered to be closest to an experimentally obtained crystal structure of an unknown material is selected from a group of a large number of obtained crystal structures, whereby the crystal structure can be identified. NPL 2 discloses a crystal structure identification technique using a density functional theory. However, the identification of a crystal structure using the density functional theory requires calculation of energy for a large number of crystal structures, which results in high computational costs and limits the number of crystal structures that can be considered, and thus it is difficult to determine a correct crystal structure.

PTL 1 discloses a method of searching for a material having predetermined characteristics by using spectrum information. More specifically, actual measurement information including synthesis conditions, physical properties, and spectrum information of a material having predetermined characteristics is acquired, and the characteristics of the material to be subjected to optimization processing are output. In PTL 1, accordingly, it is necessary to perform optimization processing using information on synthesis conditions and physical properties of a material other than spectrum information. In addition, information on a crystal structure is not included in the search result of the material to be output.

The inventors of the present application have found that an unknown crystal structure can be identified efficiently and accurately by performing an optimization step using the similarity between an experimentally obtained X-ray diffraction pattern (experimental spectrum) of an unknown material and a computationally calculated X-ray diffraction pattern (computational spectrum). Further, it has been found that it is also possible to output a structure that has a low energy and is close to an experimentally synthesized structure even by executing an optimization step using the similarity of a spectrum and energy information of a crystal structure.

Here, the spectrum refers to a group of components that can be measured with respect to the crystal structure. Examples thereof include, but are not limited to, an X-ray diffraction pattern, an X-ray absorption pattern, a nuclear magnetic resonance (NMR) spectrum, and a light absorption spectrum. The experimental spectrum is a spectrum obtained by measuring an experimentally obtained crystal structure. The computational spectrum refers to a spectrum computed from a crystal structure generated by a step of generating a crystal structure.

To address the issues described above, an information processing method according to an aspect of the present disclosure is an information processing method to be executed by a computer. The information processing method includes acquiring experimental spectrum information indicating an experimental spectrum obtained by actually measuring a material to be searched for; acquiring material information regarding a composition of the material; generating, based on the material information, pieces of candidate structure information regarding candidate structures that are candidates for a crystal structure of the material, and acquiring computational spectrum information indicating computational spectra each corresponding to a corresponding one of the candidate structures; generating structure information regarding the crystal structure, based on a correlation between the experimental spectrum information and the computational spectrum information; and outputting the generated structure information.

This makes it easy to efficiently and accurately identify an unknown crystal structure.

For example, the generating of the structure information may perform structural optimization on two or more candidate structures among the candidate structures using the experimental spectrum and the computational spectra, and the structure information may indicate a structure obtained by the structural optimization.

This makes it easy to efficiently and accurately identify an unknown crystal structure.

Further, the structural optimization may be performed using a similarity between the experimental spectrum and the computational spectra, and the structure information may indicate a structure in which the similarity is greater than or equal to a predetermined threshold among two or more structures obtained by the structural optimization.

This makes it possible to extract all candidate structures having a similarity greater than or equal to a predetermined threshold. When there are candidate structures having a similarity greater than or equal to a predetermined threshold, the structures can be compared. Thus, it is possible to identify an unknown crystal structure with higher accuracy.

Further, the structural optimization may be performed by changing at least one of a lattice constant of the candidate structures or a position of an atom in the candidate structures, and the structural optimization may be performed such that a second similarity between the experimental spectrum and a computational spectrum of a structure obtained by the structural optimization is higher than a first similarity between the experimental spectrum and a computational spectrum of the candidate structures before the structural optimization is performed.

This makes it possible to increase the accuracy of identification of an unknown crystal structure by performing structural optimization using known information of at least one of the lattice constant and the position of the atom.

Further, the structural optimization may be executed one or more times on a target candidate structure of the candidate structures until a first convergence condition is satisfied, and the first convergence condition may be at least one of the second similarity being greater than or equal to a first threshold or a difference between the second similarity and the first similarity being less than or equal to a second threshold smaller than the first threshold.

This makes it easy to efficiently and accurately identify an unknown crystal structure.

Further, the structural optimization may be performed by a gradient descent method that uses a similarity between the experimental spectrum and the computational spectra.

This makes it easy to efficiently and accurately identify an unknown crystal structure.

The information processing method may further include acquiring energy information indicating an energy calculated for each of the candidate structures. The structural optimization may be performed using the experimental spectrum information, the computational spectrum information, and the energy information.

This makes it possible to avoid the arrangement of atoms that have a high similarity but are unstable in terms of energy, and it is possible to perform structural optimization to achieve a crystal structure having a low energy and a high similarity, that is, a structure close to an unknown crystal structure of the material to be searched for.

Further, the structural optimization may be performed by a gradient descent method that uses a score obtained by combining a similarity between the experimental spectrum and the computational spectra with the energy.

This makes it possible to avoid the arrangement of atoms that have a high similarity but are unstable in terms of energy, and it is possible to perform structural optimization to achieve a crystal structure having a low energy and a high similarity, that is, a structure close to an unknown crystal structure of the material to be searched for.

Further, the structural optimization may be executed one or more times on a target candidate structure of the candidate structures until a second convergence condition is satisfied, and the second convergence condition may be a predetermined condition based on the score.

This makes it possible to avoid the arrangement of atoms that have a high similarity but are unstable in terms of energy, and it is possible to perform structural optimization to achieve a crystal structure having a low energy and a high similarity, that is, a structure close to an unknown crystal structure of the material to be searched for.

Further, the experimental spectrum and the computational spectra may be spectra obtained by an X-ray diffraction method.

This makes it easy to reproduce a crystal structure that is a three-dimensional periodic structure with high accuracy.

Further, an information processing method according to an aspect of the present disclosure is an information processing method to be executed by a computer. The information processing method includes generating structure information regarding a crystal structure of a material to be searched for, based on a correlation between experimental spectrum information and computational spectrum information, the experimental spectrum information indicating an experimental spectrum obtained by actually measuring the material, the computational spectrum information indicating a computational spectrum calculated for each of candidate structures that are candidates for the crystal structure; and outputting the generated structure information.

This makes it easy to efficiently and accurately identify an unknown crystal structure.

An information processing system according to an aspect of the present disclosure includes a display that displays a first image for receiving an input of material information regarding a composition of a material to be searched for and experimental spectrum information indicating an experimental spectrum obtained by actually measuring the material; and a display controller that causes the display to display a second image generated based on the material information and the experimental spectrum information that are input and representing structure information regarding a crystal structure of the material.

This makes it easy for a user to confirm an efficient and accurate identification result of an unknown crystal structure. It is also possible for the user to identify an unknown crystal structure by utilizing the knowledge of the user themselves.

A non-transitory computer-readable recording medium stores a program for causing a computer to execute processing including acquiring experimental spectrum information indicating an experimental spectrum obtained by actually measuring a material to be searched for; acquiring material information regarding a composition of the material; generating, based on the material information, pieces of candidate structure information regarding candidate structures that are candidates for a crystal structure of the material, and acquiring computational spectrum information indicating computational spectra each corresponding to a corresponding one of the candidate structures; generating structure information regarding the crystal structure, based on a correlation between the experimental spectrum information and the computational spectrum information; and outputting the generated structure information.

This makes it easy to efficiently and accurately identify an unknown crystal structure.

Further, the present disclosure can be implemented as a computer program for causing a computer to execute characteristic processing included in an information processing method according to the present disclosure. It goes without saying that the computer program can be distributed via a computer-readable non-transitory recording medium such as a CD-ROM or a communication network such as the Internet.

Hereinafter, embodiments will be described in detail with reference to the drawings.

It should be noted that each of the following embodiments represents a comprehensive or specific example of the present disclosure. Numerical values, shapes, materials, components, the arrangement and connection of the components, steps, the processing order of the steps, and the like described in the following embodiments are examples and are not intended to limit the present disclosure. Among the components of the following embodiments, components not recited in any one of the independent claims that indicate the broadest concepts are described as optional components. In all of the embodiments, the respective contents may be combined. The drawings are schematic and not necessarily drawn to scale. In the drawings, the same elements are denoted by the same reference numerals.

An information processing system according to embodiments of the present disclosure may be configured such that all the components thereof are included in one computer, or may be configured as a system whose components are distributed to computers.

### Embodiment 1

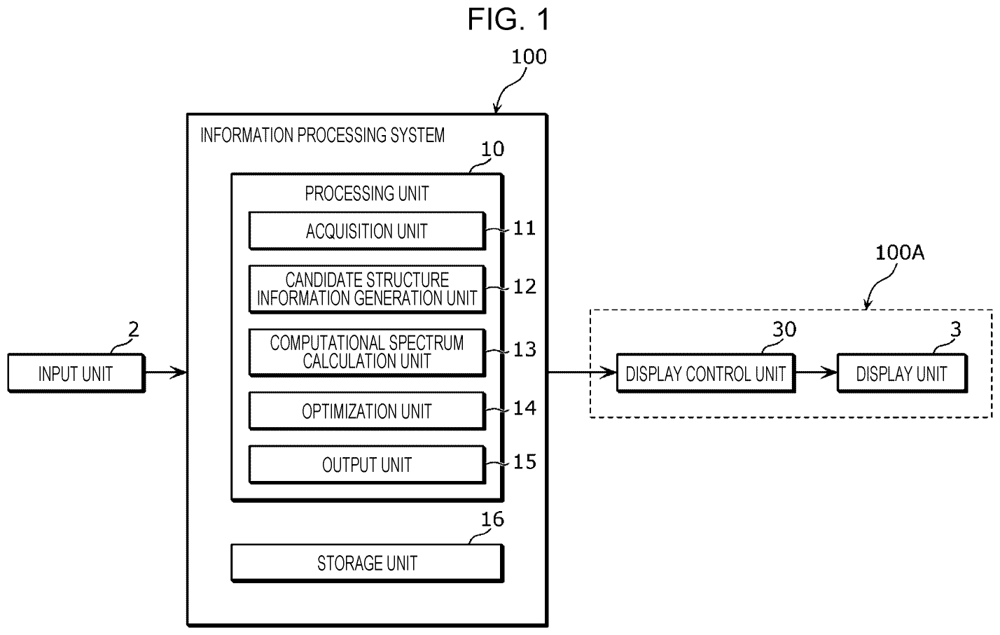

Hereinafter, an information processing system 100 (information processing method or program) according to Embodiment 1 of the present disclosure will be described in detail with reference to the drawings. FIG. 1 is a block diagram illustrating an overall configuration including the information processing system 100 according to Embodiment 1. The information processing system 100 is configured as, for example, a computer such as a personal computer or a server. That is, the information processing system 100 may be implemented by, for example, cloud computing. In the description of Embodiment 1, the information processing system 100 is a stationary computer.

The information processing system 100 includes a processing unit 10 and a storage unit 16. The processing unit 10 includes an acquisition unit 11, a candidate structure information generation unit 12, a computational spectrum calculation unit 13, an optimization unit 14, and an output unit 15. An input unit 2, a display control unit 30, and a display unit 3 are connected to the information processing system 100. The input unit 2, the display control unit 30, and the display unit 3 are configured by an information terminal used by a user, such as a smartphone, a tablet terminal, or a personal computer.

The input unit 2 is an input interface that receives an input from the user, and includes, for example, a keyboard, a touch sensor, a touch pad, a mouse, or the like. The input unit 2 receives an input operation by the user and outputs a signal corresponding to the input operation to the information processing system 100. In the present disclosure, the display unit 3 and the input unit 2 are configured independently of each other, but may be configured into a single unit such as a touch panel. In the present disclosure, the information processing system 100 does not include the display unit 3 and the input unit 2, but may include them.

The input unit 2 receives input of material information regarding a material to be searched for and experimental spectrum information indicating an experimental spectrum obtained by actually measuring the material to be searched for. In Embodiment 1, the material information is a composition formula of the material to be searched for.

In Embodiment 1, the experimental spectrum is an X-ray diffraction pattern obtained by performing an X-ray diffraction method on the material to be searched for. Likewise, a computational spectrum described below is also an X-ray diffraction pattern. The X-ray diffraction pattern is characterized by a three-dimensional periodic atomic array pattern. Accordingly, structural optimization (described below) is performed so that the coincidence (that is, the similarity) between the experimental spectrum and the computational spectrum becomes high, thereby making it possible to obtain a structure in which a three-dimensional periodic atomic array, that is, a crystal structure, is similar to the experimentally obtained crystal structure.

The display control unit 30 causes the display unit 3 to display an image or the like based on information output from the output unit 15 of the information processing system 100.

The display unit 3 is controlled by the display control unit 30 to display an image or the like. The display unit 3 is, for example, but not limited to, a liquid crystal display, a plasma display, or an organic electro-luminescence (EL) display.

The acquisition unit 11 acquires material information and experimental spectrum information. The acquisition unit 11 is a subject that executes a step of acquiring material information and a step of acquiring experimental spectrum information in an information processing method according to the present disclosure. Specifically, the acquisition unit 11 acquires material information and experimental spectrum information that are input by the user through the input unit 2. For example, the user performs an operation of inputting material information and experimental spectrum information while viewing a first image displayed on the display unit 3 to receive input of the material information and the experimental spectrum information.

The candidate structure information generation unit 12 generates, based on the material information acquired by the acquisition unit 11, candidate structure information regarding candidate structures that are candidates for a crystal structure of the material. The candidate structure information generation unit 12 is a subject that executes part of a step of acquiring computational spectrum information in the information processing method according to the present disclosure. Details of the processing executed by the candidate structure information generation unit 12 will be described below.

The computational spectrum calculation unit 13 calculates a computational spectrum corresponding to each of the candidate structures. The computational spectrum calculation unit 13 is a subject that executes the step of acquiring computational spectrum information in the information processing method according to the present disclosure. Details of the processing executed by the computational spectrum calculation unit 13 will be described below.

The optimization unit 14 generates structure information regarding a crystal structure of the material to be searched for, based on the correlation between the experimental spectrum information acquired by the acquisition unit 11 and the computational spectrum information calculated by the computational spectrum calculation unit 13. The optimization unit 14 is a subject that executes a step of generating structure information in the information processing method according to the present disclosure.

In Embodiment 1, the optimization unit 14 performs structural optimization on at least one candidate structure among the candidate structures by using an experimental spectrum and a computational spectrum. The structure information indicates one or more structures obtained by the structural optimization. Here, a structure obtained by the structural optimization is considered to be a structure that is the same as or close to an unknown crystal structure of the material to be searched for. In other words, a structure obtained by the structural optimization is a globally and locally stable structure. More specifically, the structural optimization is performed by a gradient descent method that uses the similarity between the experimental spectrum and the computational spectrum. The structure information indicates a structure having the highest similarity among the one or more structures obtained by the structural optimization, or, in other words, a structure considered to be closest to an unknown crystal structure of the material to be searched for. Details of the structural optimization will be described below.

The output unit 15 outputs an image or the like to the display control unit 30 to cause the display unit 3 to display the image or the like. The output unit 15 also outputs the structure information generated by the optimization unit 14. The output unit 15 is a subject that executes a step of outputting structure information in the information processing method according to the present disclosure. Specifically, the output unit 15 causes the display unit 3 to display a second image representing the structure information generated by the optimization unit 14 to output the structure information.

The storage unit 16 is a recording medium that stores the material information and the experimental spectrum information, which are acquired by the acquisition unit 11, pieces of candidate structure information regarding the candidate structures generated by the candidate structure information generation unit 12, the computational spectrum information calculated by the computational spectrum calculation unit 13, the structure information regarding the crystal structure generated by the optimization unit 14, and the like. The recording medium is, for example, a hard disk drive, a random access memory (RAM), a read only memory (ROM), a semiconductor memory, or the like. Such a recording medium may be volatile or non-volatile.

### Operation

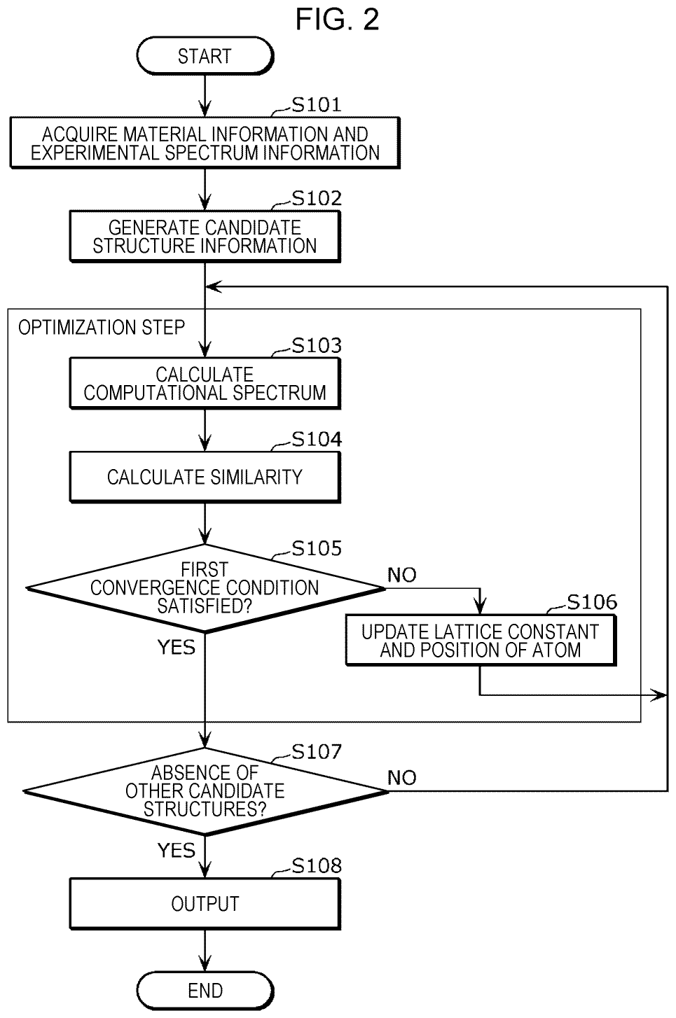

Hereinafter, the operation of the information processing system 100 (that is, an information processing method) according to Embodiment 1 will be described. FIG. 2 is a flowchart illustrating an overview of the operation of the information processing system 100 according to Embodiment 1.

### Step S101

The acquisition unit 11 acquires material information and experimental spectrum information. In step S101 (input step), a composition formula of a material to be searched for and a measured experimental spectrum are input. The composition formula of the material to be searched for may be a composition formula expected from the raw material or a composition formula determined by elementary analysis. When the total value (=a+b+ . . . ) of the number of all the elements included in the composition formula AaBb . . . is relatively large, the crystal structure corresponding to the composition formula is complicated. A is an element, a is the number of elements A, B is an element, b is the number of elements B, etc., with a being an integer greater than or equal to 1, b being an integer greater than or equal to 1, etc. In the information processing method according to the present disclosure, it is possible to handle a composition formula in which the total value exceeds 100. An example of the composition formula in which the total value exceeds 100 is Ca₄₀Ti₄₀O₁₂₀. The above-described composition formula in which the total value is relatively large is handled by the density functional theory, resulting in high computational cost. That is, the density functional theory cannot realistically handle a composition formula in which the total value is relatively large. In the information processing method according to the present disclosure, by contrast, Embodiment 1 does not involve energy computation, and Embodiment 2, which will be described below, does not necessarily require the density functional theory for energy computation. Thus, the information processing method can handle the above-described composition formula in which the total value is relatively large.

When the composition formula of the material to be searched for is unknown, it is possible to input composition formulas. In this case, in the information processing method according to the present disclosure, it is possible to perform structural optimization on each of the input composition formulas and output a structure having the highest coincidence among the obtained structures as final structure information. The input composition formulas and the obtained structures may have a one-to-one correspondence. In the description of the following process, one composition formula is input in S101.

### Step S102

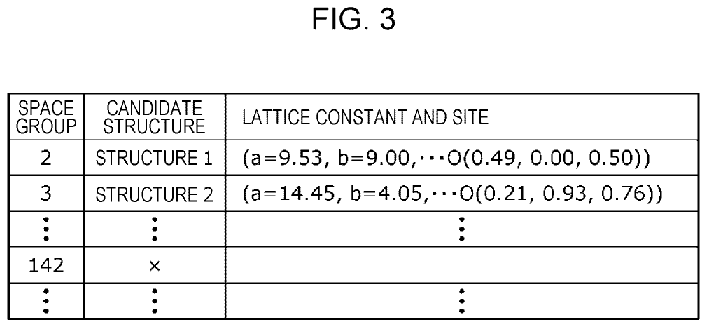

The candidate structure information generation unit 12 generates pieces of candidate structure information regarding candidate structures, based on the material information acquired by the acquisition unit 11. In step S102 (candidate structure information generation step), for example, pieces of candidate structure information regarding candidate structures are generated using space groups used to describe the symmetry of a three-dimensional structure, based on the composition formula of the material to be searched for acquired by the acquisition unit 11. FIG. 3 is a diagram illustrating an example of a list of pieces of candidate structure information. The list illustrated in FIG. 3 includes, in order from the left, a column indicating a space group, a column indicating the number of a candidate structure, and a column indicating lattice constants and sites (that is, positions to be occupied by atoms in a crystal structure) in the candidate structure. FIG. 3 illustrates a list of pieces of candidate structure information when the composition formula of the material to be searched for is “Ca₄Ti₄O₁₂”. In an example illustrated in FIG. 3, candidate structure information corresponding to the space group numbers “2” and “3” can be generated, but candidate structure information corresponding to the space group number “142” cannot be generated. The candidate structures and the pieces of candidate structure information have a one-to-one correspondence. Each of the pieces of candidate structure information may include information indicating lattice constants and information indicating sites in the candidate structure.

The candidate structure information generation unit 12 generates candidate structure information when all the atoms included in the composition formula of the material to be searched for can be arranged at any site in the crystal structure indicated by the space group, and does not generate candidate structure information when all the atoms cannot be arranged. Then, the candidate structure information generation unit 12 performs the above-described process on all the space groups to generate pieces of candidate structure information regarding candidate structures.

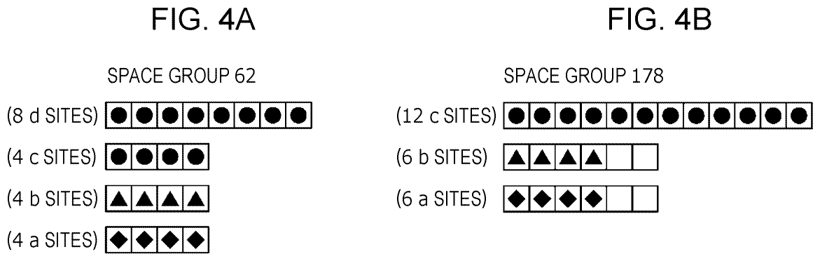

FIGS. 4A and 4B are explanatory diagrams of a method of generating candidate structure information. The arrangement of atoms included in the composition formula is determined under constraints in which the same atom is arranged at all t sites in the crystal structure and the number of the t sites is s. In FIGS. 4A and 4B, a circle, a triangle, and a rhombus represent different kinds of atoms. FIG. 4A illustrates four a sites, four b sites, four c sites, and eight d sites included in the crystal structure represented by the space group number “62”. That is, (s, t)=(4, a), (4, b), (4, c), (8, d). When the composition formula of the material to be searched for is “Ca₄Ti₄O₁₂”, four Ca atoms, four Ti atoms, and twelve O atoms can be arranged in the crystal structure under the above-described constraints. Thus, the candidate structure information generation unit 12 can generate the candidate structure information corresponding to the space group number “62”. FIG. 4B illustrates six a sites, six b sites, and twelve c sites included in the crystal structure represented by the space group number “178”. That is, (s, t)=(6, a), (6, b), (12, c). When the composition formula of the material to be searched for is “Ca₄Ti₄O₁₂”, four Ca atoms, four Ti atoms, and twelve O atoms cannot be arranged in the crystal structure under the above-described constraints. Thus, the candidate structure information generation unit 12 cannot generate the candidate structure information corresponding to the space group number “178”.

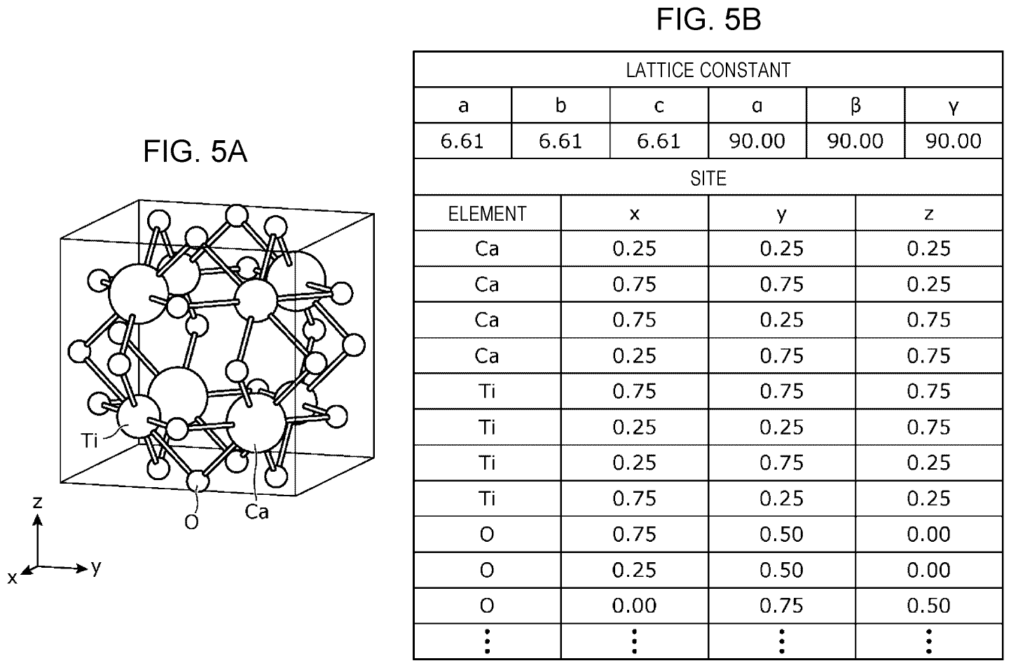

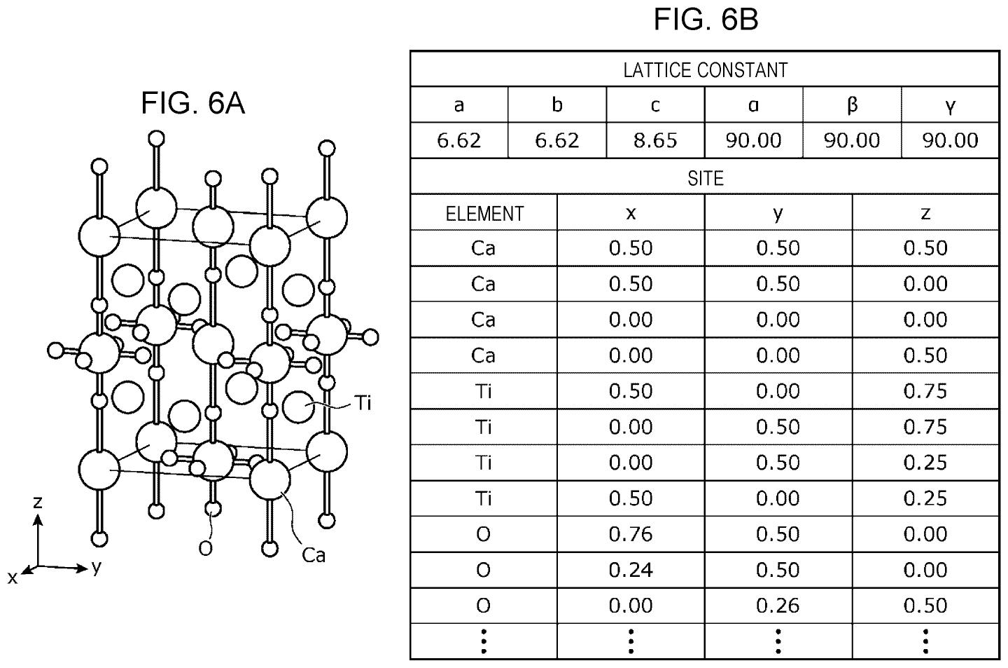

FIGS. 5A and 5B are diagrams illustrating a specific example of the candidate structure information. FIGS. 6A and 6B are diagrams illustrating another specific example of the candidate structure information. FIGS. 5A and 6A are diagrams illustrating examples of candidate structure information when the composition formula of the material to be searched for is “Ca₄Ti₄O₁₂”. FIGS. 5B and 6B are diagrams illustrating examples of lattice constants and sites in candidate structures when the composition formula of the material to be searched for is “Ca₄Ti₄O₁₂”.

The candidate structure information generation unit 12 may generate pieces of candidate structure information by, instead of the above-described method, a Markov chain Monte Carlo method, for example. This method enables generation of candidate structure information using an energy-based probability density. In each step of the Markov chain Monte Carlo method, the position of an atoms or a crystal lattice parameter is changed. At this time, only one or multiple of the position of an atom or a crystal lattice parameter may be changed. In this method, the energy is evaluated before and after the change, and a decision is made to accept or reject the change. The above-described step is repeated, and one piece of candidate structure information is generated, for example, every several hundreds to several thousands of times of repetition. Thus, it is possible to generate candidate structure information from the energy-based probability density.

Further, in the Markov chain Monte Carlo method, controlling the rejection probability by introducing a temperature term makes it possible to control whether to generate a large number of pieces of locally stable candidate structure information or various kinds of candidate structure information. For example, when information on the crystal structure of the material to be searched for is obtained even partially, it is possible to efficiently identify an unknown crystal structure by generating a large number of pieces of locally stable candidate structure information. On the other hand, when there is no information on the crystal structure of the material to be searched for, it is possible to select a crystal structure considered to be optimal from a larger number of candidate structures by giving priority to generation of various kinds of candidate structure information. As described above, in the generation of candidate structure information by the Markov chain Monte Carlo method, switching the mode in accordance with whether there is information on the crystal structure of the material to be searched for enables an efficient search for an unknown crystal structure.

The candidate structure information generation unit 12 may generate pieces of candidate structure information by, for example, a genetic algorithm. This method enables efficient generation of various kinds of candidate structure information. The genetic algorithm generates a child structure from a parent structure. Generally, the initial parent structure is a random structure in which atoms are randomly arranged in a randomly generated crystal lattice. In the genetic algorithm, a child structure is generated by a mutation or crossover operation using one or two parent structures. For example, the mutation operation is carried out by exchanging the positions of atoms, and the crossover operation is carried out by, for example, dividing and combining two structures. Both the mutation operation and the crossover operation have a small computation load and can greatly change a structure at a time. Thus, generation of candidate structure information using the genetic algorithm makes it possible to efficiently generate various kinds of candidate structure information.

The candidate structure information generation unit 12 may generate pieces of candidate structure information randomly, or may generate pieces of candidate structure information by using information on another crystal structure.

Subsequent steps S103 to S106 correspond to an optimization step. In the optimization step, structural optimization is performed on each of the candidate structures corresponding to the pieces of candidate structure information generated in S102. Here, the structural optimization of a candidate structure means to optimize a lattice constant of the candidate structure and the coordinates (position) of each atom in the candidate structure. In other words, the structural optimization is performed by changing at least one of a lattice constant of a target candidate structure and the position of an atom in the candidate structure. The coordinates of each atom are represented by, for example, an orthogonal coordinate system, fractional coordinates with respect to a lattice, or polar coordinates. The coordinates of each atom may be determined by defining a multi-dimensional space to which information on the element itself is added, in addition to spatial information in the crystal.

The structural optimization described above may be performed by “changing a lattice constant of the candidate structure”, “changing the position of an atom in the candidate structure”, or “changing a lattice constant of the candidate structure and changing the position of an atom in the candidate structure”

In the information processing system 100 (information processing method) according to Embodiment 1, the optimization step is performed by a gradient descent method that uses the similarity between the experimental spectrum and the computational spectrum. That is, in the information processing system 100 according to Embodiment 1, the structural optimization of the candidate structure is performed so that the experimental spectrum and the computational spectrum coincide with each other. The similarity between the spectra is evaluated by an error index or a similarity index.

Here, the error index is an index for evaluating an error between spectra, and the smaller the value is, the higher the similarity is evaluated. The error index is obtained by evaluating, for example, a Euclidean distance, a Mahalanobis distance, a Manhattan distance, a Chebyshev distance, a Minkowski distance, or the like for each measurement point of each spectrum. It is also possible to convert these distances into a mean absolute error (MAE), a mean square error (MSE), a root mean square error (RMSE), a root mean square percentage error (RMSPE), a mean absolute percentage error (MAPE), or the like and calculate one index for each pair of spectra.

The similarity index is an index for evaluating the similarity between spectra, and the larger the value is, the higher the similarity is evaluated. As the similarity index, cosine similarity, Pearson's correlation coefficient, deviation pattern similarity, or the like can be used.

In the optimization step, only one of these error indices and similarity indices may be used, or multiple indices may be used. When multiple indices are used, the similarity can be evaluated by a weighted average over the multiple indices each given a coefficient. In Embodiment 1, the index (similarity) used in the optimization step is the cosine similarity.

### Step S103

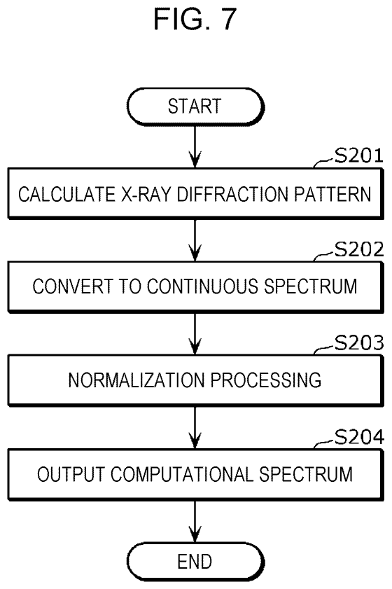

The computational spectrum calculation unit 13 calculates a computational spectrum for one candidate structure (or structurally optimized structure) among the candidate structures corresponding to the pieces of candidate structure information generated by the candidate structure information generation unit 12. A procedure for calculating a computational spectrum will be described by taking, as an example, a case where an X-ray diffraction pattern is used in step S103 (computational spectrum calculation step). FIG. 7 is a flowchart illustrating an example of computational spectrum calculation.

### Step S201

The computational spectrum calculation unit 13 calculates an X-ray diffraction pattern of the candidate structure (or structurally optimized structure). The intensity of the X-ray diffraction pattern can be determined by Equation (1) below for a crystal plane defined by the Miller indices h, k, and 1. In Equation (1), dₕₖₗ is the crystal plane distance, and the relationship between dₕₖₗ and the Bragg angle θ can be calculated in accordance with Bragg's equation, which is given by Equation (2), by using the wavelength λ of the X-ray used.

$$F ( h k l ) = {\sum }_{j = 1}^{N} {f}_{j} exp \{ 2 π i ( h {x}_{j} + k {y}_{j} + l {z}_{j} ) \} \qquad ( 1 )$$

$$2 {d}_{h k l} sin θ = n λ ( n = 1 , 2 , 3 … ) \qquad ( 2 )$$

In Equation (1), “h”, “k”, and “1” each represent an element of a lattice plane (hkl), “fⱼ” represents an atomic scattering factor of an atom j, “xⱼ”, “yⱼ”, and “zⱼ” represent an x coordinate, a y coordinate, and a z coordinate of the atom j, respectively, and “F(hkl)” represents a crystal structure factor. Since the square of the crystal structure factor coincides with the peak intensity, the diffraction intensity of hkl reflection can be calculated from the coordinates of the atom and the kind of the element. That is, the X-ray diffraction pattern is a spectrum well reflecting the arrangement of atoms and the kinds of elements in the crystal structure, and is a spectrum useful for identifying the crystal structure.

### Step S202

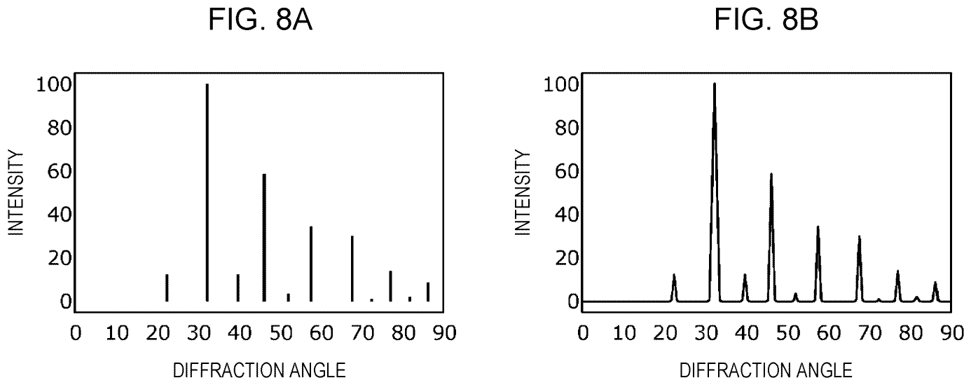

Next, the computational spectrum calculation unit 13 converts a discrete spectrum of the obtained X-ray diffraction pattern into a continuous spectrum. The conversion from the discrete spectrum to the continuous spectrum can be performed by applying a Gauss function, a Lorentz function, a Pseudo-Voigt function, or the like. FIGS. 8A and 8B are diagrams illustrating an example of the discrete spectrum and the continuous spectrum. FIG. 8A illustrates a discrete spectrum of an X-ray diffraction pattern, and FIG. 8B illustrates a continuous spectrum obtained by converting the discrete spectrum illustrated in FIG. 8A using a Gauss function (standard deviation: 0.3). In FIGS. 8A and 8B, the vertical axis represents intensity, and the horizontal axis represents 2θ(“2θ” is a diffraction angle). The unit of 2θ is an angle.

In the optimization step, as described below, the similarity between the computational spectrum and the experimental spectrum, both of which are continuous spectra, is calculated. In this case, it is desirable to remove the background of the experimental spectrum. This is because the removal of the background leads to more accurate computation of the similarity between the computational spectrum and the experimental spectrum. It is also possible to convert the experimental spectrum, which is a continuous spectrum, into a discrete spectrum once and then convert it into a continuous spectrum again before computing the similarity. In this case, the conversion of the experimental spectrum into a continuous spectrum and the conversion of the computational spectrum into a continuous spectrum are performed under the same conditions, thus making it possible to compute the similarity more accurately. This method enables similarity computation that is less likely to depend on noise in the measurement of the experimental spectrum.

In the optimization step, in the calculation of the similarity between the computational spectrum and the experimental spectrum, both of which are discrete spectra, it is desirable to extract a peak position and intensity before converting the experimental spectrum into a discrete spectrum. At this time, all of the peaks may be converted, or only some of the peaks may be converted. For example, when the noise is large in the measurement of the experimental spectrum, it is possible to suppress the influence of the noise by extracting only a peak having an intensity greater than or equal to a certain value.

### Step S203

Next, the computational spectrum calculation unit 13 performs normalization processing on the converted continuous spectrum of the computational spectrum. As a result, the intensity of the continuous spectrum of the computational spectrum is normalized.

### Step S204

Then, the computational spectrum calculation unit 13 outputs the normalized computational spectrum. The output computational spectrum is used to calculate the similarity in the optimization step.

Steps S201 to S204 described above are executed as step S103.

### Step S104

Returning to FIG. 2, next, the optimization unit 14 calculates the similarity between the experimental spectrum, which is the measured spectrum acquired by the acquisition unit 11, and the computational spectrum, which is calculated by the computational spectrum calculation unit 13. As described above, in Embodiment 1, the optimization unit 14 evaluates the similarity between the experimental spectrum and the computational spectrum by using the cosine similarity, which is a similarity index. Accordingly, the similarity is represented by a value of 0 to 1, and the larger the numerical value is, the more similar the experimental spectrum and the computational spectrum are.

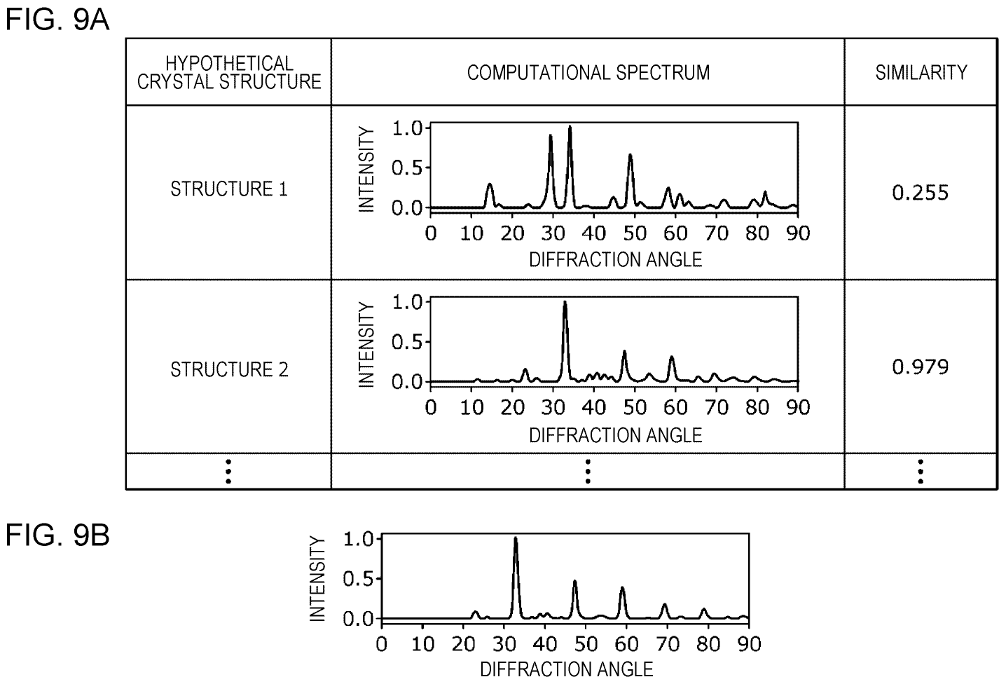

FIGS. 9A and 9B are explanatory diagrams of the similarity between the experimental spectrum and the computational spectrum. A table illustrated in FIG. 9A includes, in order from the left, a column indicating the number of a candidate structure, a column indicating a computational spectrum, and a column indicating similarity. FIG. 9B illustrates an experimental spectrum. In an example illustrated in FIG. 9A, the similarity between the computational spectrum of the candidate structure “Structure 1” and the experimental spectrum is relatively low, and the similarity between the computational spectrum of the candidate structure “Structure 2” and the experimental spectrum is relatively high. In the diagram illustrating the computational spectra illustrated in FIG. 9A and the diagram illustrating the experimental spectrum illustrated in FIG. 9B, the vertical axis represents intensity, and the horizontal axis represents 2θ(“2θ” is a diffraction angle). The unit of 20 is an angle.

### Step S105

Next, the optimization unit 14 determines whether the calculated similarity satisfies a first convergence condition. The first convergence condition is a condition in which the calculated similarity is greater than or equal to a first threshold (for example, 0.99) in a case where the structural optimization has not been executed even one time. On the other hand, in a case where the structural optimization has been executed one or more times, the first convergence condition is at least one of second similarity being greater than or equal to the first threshold and the difference between the second similarity and first similarity being less than or equal to a second threshold (for example, 0.01) smaller than the first threshold. The first convergence condition in a case where the structural optimization has been executed one or more times may be “the second similarity being greater than or equal to a first threshold”, “the difference between the second similarity and the first similarity being less than or equal to a second threshold smaller than the first threshold”, or “the second similarity being greater than or equal to the first threshold and the difference between the second similarity and the first similarity being less than or equal to the second threshold”.

The first similarity is the similarity between the experimental spectrum and the computational spectrum of the candidate structure before the structural optimization is performed. The second similarity is the similarity between the experimental spectrum and the computational spectrum of a structure obtained by the structural optimization. In other words, the second similarity is the similarity between the experimental spectrum and the computational spectrum calculated for the structure obtained after a lattice constant and the position of an atom of the candidate structure are updated in step S106 described below. In a case where the structural optimization has been executed multiple times, the second similarity is the similarity for the latest structurally optimized structure, and the first similarity is the similarity for the structure structurally optimized immediately before the latest structural optimization has been executed.

If the similarity satisfies the first convergence condition (step S105: Yes), then, the optimization unit 14 executes step S107. On the other hand, if the similarity does not satisfy the first convergence condition (step S105: No), then, the optimization unit 14 executes step S106. That is, when the similarity for the candidate structure or a structure obtained by structurally optimizing the candidate structure is greater than or equal to the first threshold, the optimization step ends. When the difference between the second similarity and the first similarity is less than or equal to the second threshold (in other words, a gradient described below is less than or equal to a threshold) for the candidate structure or a structure obtained by structurally optimizing the candidate structure, the optimization step ends. The upper limit number of times the optimization step is to be repeated may be set. In this case, at the point in time when the number of times the optimization step is repeated reaches the upper limit, the process can proceed to S107.

In this way, the structural optimization is executed one or more times on the target candidate structure until the first convergence condition is satisfied or the upper limit number of times the optimization step is to be repeated is reached. When the similarity for the candidate structure satisfies the first convergence condition, in some cases, the structural optimization is not executed even one time.

### Step S106

Next, the optimization unit 14 updates a lattice constant and the position of an atom of the candidate structure (or structurally optimized structure). First, the optimization unit 14 calculates a gradient with respect to the similarity to change a lattice constant and the position of an atom of the candidate structure (or structurally optimized structure) so that the similarity improves (increases when a similarity index is used, and decreases when an error index is used). The gradient can be calculated by partially differentiating the similarity with respect to each component of the coordinates of an atom and a lattice constant. Then, the optimization unit 14 applies an optimization algorithm described below using the calculated gradient to update a lattice constant and the position of an atom of the candidate structure (or structurally optimized structure). In Embodiment 1, both a lattice constant and the position of an atom are updated. Either of them may be updated. The update of the positions of the atoms may be performed by updating the positions of all the atoms or updating only the position of any one of the atoms. Then, the information processing system 100 (information processing method) returns to step S103, and executes the optimization step again.

As the optimization algorithm, a steepest descent method, a Newton method, a quasi-Newton method, a conjugate gradient method, a derivative of these methods, or the like can be used. As the optimization algorithm, a moving average of the gradient may be used, or an algorithm such as Adagrad, Adadelta, or Adam that adaptively changes the learning rate in accordance with a change in the gradient may be used.

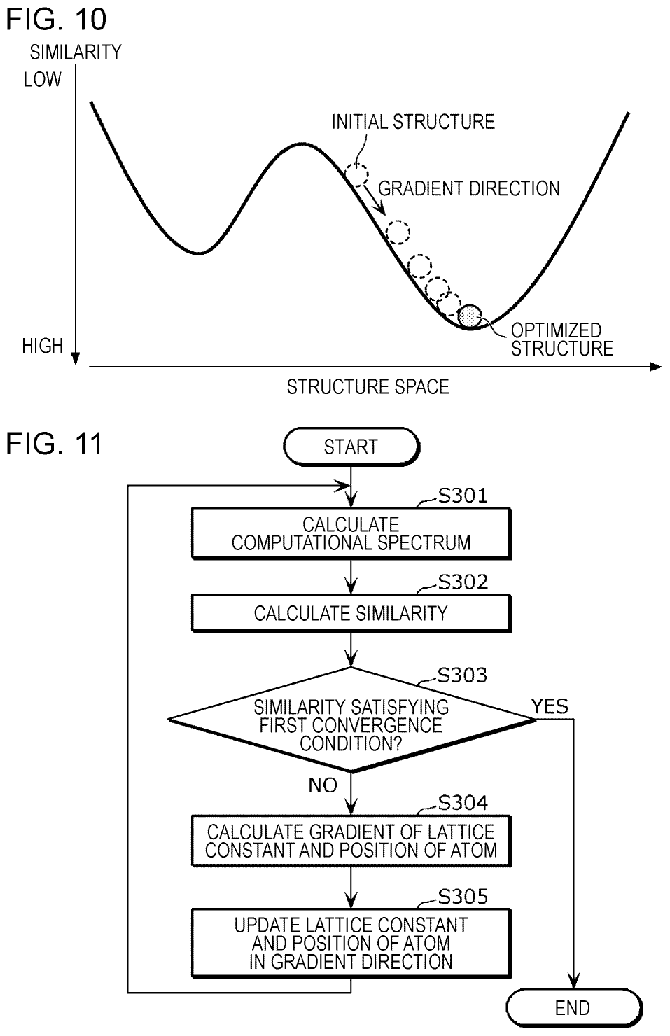

FIG. 10 is an explanatory diagram of structural optimization by the information processing system 100 according to Embodiment 1. In FIG. 10, the vertical axis represents similarity, and the horizontal axis represents structure space. In an example illustrated in FIG. 10, an initial structure obtained before the structural optimization is executed is located in the middle of a similarity surface and is an unstable structure with a relatively low similarity. By contrast, an optimized structure obtained after the structural optimization is executed one or more times along a gradient direction is located in a valley of the similarity surface, and is a locally stable structure with a relatively high similarity.

As described above, the structural optimization is executed along the gradient direction (that is, the direction in which the similarity improves) so that the gradient becomes less than or equal to the threshold. In other words, the structural optimization is performed so that the second similarity between the experimental spectrum and the computational spectrum of a structure obtained by the structural optimization is higher than the first similarity between the experimental spectrum and the computational spectrum of the candidate structure before the structural optimization is performed.

The above-described optimization step is illustrated again in FIG. 11. FIG. 11 is a flowchart illustrating an example of the optimization step performed by the information processing system 100 according to Embodiment 1.

### Step S301

First, the computational spectrum calculation unit 13 calculates a computational spectrum for one candidate structure (or structurally optimized structure) among the candidate structures corresponding to the pieces of candidate structure information generated by the candidate structure information generation unit 12. Step S301 is the same processing as step S103 (see FIG. 2).

### Step S302

Next, the optimization unit 14 calculates the similarity between the experimental spectrum acquired by the acquisition unit 11 and the computational spectrum calculated by the computational spectrum calculation unit 13. Step S302 is the same processing as step S104 (see FIG. 2).

### Step S303

Next, the optimization unit 14 determines whether the calculated similarity satisfies a first convergence condition. Step S303 is the same processing as step S105 (see FIG. 2). If the similarity satisfies the first convergence condition (step S303: Yes), the optimization step ends. On the other hand, if the similarity does not satisfy the first convergence condition (step S303: No), then, the optimization unit 14 executes step S304.

### Step S304

Next, the optimization unit 14 calculates a gradient of a lattice constant and the position of an atom of the candidate structure (or structurally optimized structure). Step S304 is the same processing as part of step S106 (see FIG. 2).

### Step S305

Next, the optimization unit 14 changes a lattice constant and the position of an atom of the candidate structure (or structurally optimized structure) along the calculated gradient direction to update the lattice constant and the position of the atom. Step S305 is the same processing as part of step S106 (see FIG. 2). Then, the information processing system 100 (information processing method) returns to step S301, and executes the optimization step again.

### Step S107

Returning to FIG. 2, the optimization unit 14 determines whether any other candidate structure is present. If any other candidate structure is present (step S107: No), the process returns to step S103, and the optimization step is executed on any other candidate structure. On the other hand, if no other candidate structure is present (step S107: Yes), then, the information processing system 100 (information processing method) executes step S108.

### Step S108

The output unit 15 outputs the structure information generated by the optimization unit 14. In step S108 (output step), structure information obtained in the optimization step is output. The structure information includes information regarding lattice constants and the positions of atoms of one or more structures generated by the optimization unit 14. The output format of the structure information is not particularly limited, and may be, for example, a format in which parameters are simply listed or a standardized format such as a Crystallographic Information File (CIF). Here, the output unit 15 causes the display unit 3 to display the second image representing the structure information generated by the optimization unit 14 to output the structure information.

The output unit 15 may output one or more pieces of structure information determined in the optimization step. The output unit 15 may cause the display unit 3 to display the one or more pieces of structure information. Each of the one or more pieces of structure information may correspond to one piece of material information input in step S101, that is, one composition formula. In step S108 (output step), the one or more pieces of structure information may be output. Each of the one or more pieces of structure information may include information regarding lattice constants and the positions of atoms that determine each of one or more candidate structures that are one or more candidates of the crystal structure corresponding to the composition formula.

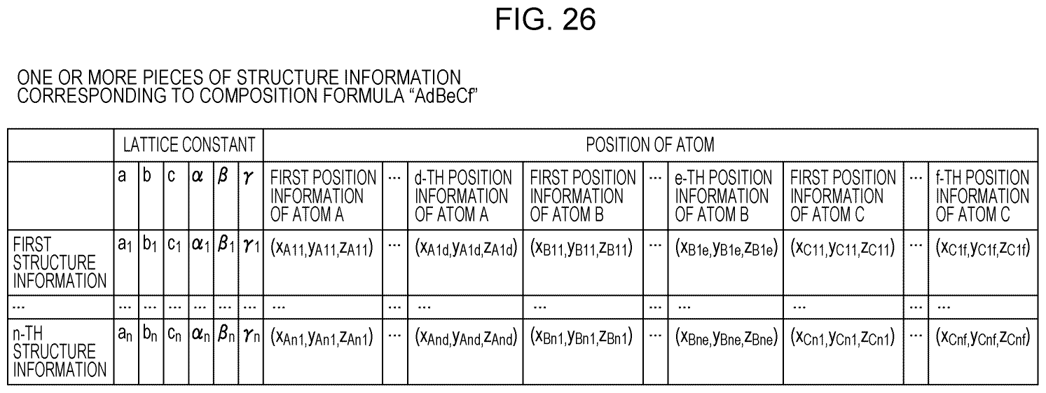

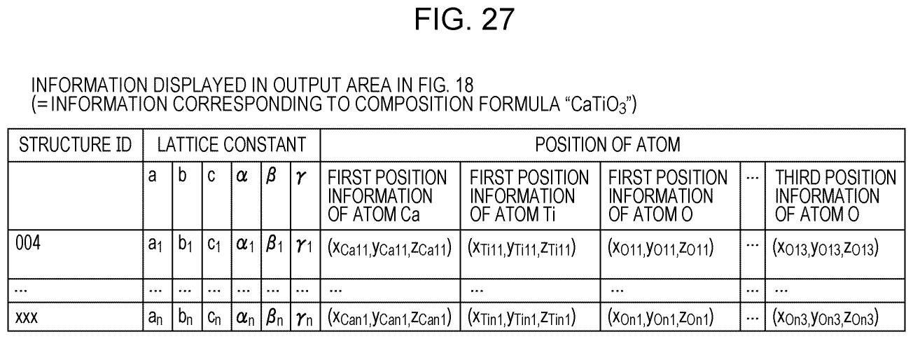

FIG. 26 is a diagram illustrating an example of one or more pieces of structure information. The one or more pieces of structure information are first structure information, . . . , and n-th structure information, where n is a natural number greater than or equal to 1.

The lattice constants include respective lengths of sides a, b, and c of the unit lattice, an angle γ formed by the sides a and b, an angle α formed by the sides b and c, and an angle β formed by the sides c and a. In FIG. 26, the length of the side a is shown in column a, the length of the side b is shown in column b, and the length of the side c is shown in column c. In FIG. 26, the angle α is shown in column α, the angle β is shown in column β, and the angle γ is shown in column γ.

The positions of atoms are indicated by three-dimensional coordinates (x, y, z). In FIG. 26, the first structure information includes, as atomic position information, three-dimensional coordinates of d atoms A, three-dimensional coordinates of e atoms B, three-dimensional coordinates of f atoms C, etc., and the n-th structure information includes, as atomic position information, three-dimensional coordinates of d atoms A, three-dimensional coordinates of e atoms B, and three-dimensional coordinates of f atoms C. The three-dimensional coordinates of the d atoms A included in the first structure information are (x_{A11}, y_{A11}, z_{A11}) to (x_{A1d}, y_{A1d}, z_{A1d}).

As described above, in Embodiment 1, the optimization step is performed using the correlation (similarity) between an X-ray diffraction pattern (experimental spectrum) obtained by actually measuring an unknown material to be searched for and an X-ray diffraction pattern (computational spectrum) calculated for each of candidate structures that are candidates for a crystal structure of the material. In Embodiment 1, therefore, it is easy to efficiently and accurately identify an unknown crystal structure.

### Embodiment 2

Hereinafter, an overview of an information processing system 200 (information processing method or program) according to Embodiment 2 of the present disclosure will be described first. In the information processing system 200 (information processing method) according to Embodiment 2, an optimization technique using both spectra and energy facilitates efficient and accurate identification of an unknown crystal structure. The differences between this optimization technique and the conventional techniques will be described hereinafter.

The conventional Rietveld method computes an X-ray diffraction pattern of a candidate structure by using, as an input, candidate structure information regarding a candidate structure created by an analyst. Further, the computed X-ray diffraction pattern is compared with an experimentally obtained X-ray diffraction pattern, and an error is computed. Then, based on the obtained error, the analyst corrects the candidate structure information regarding the candidate structure and inputs the corrected candidate structure information again. The series of processes described above is repeated until the error is acceptable to identify the crystal structure.

In the identification of a crystal structure by the conventional density functional theory, an analyst prepares a large number of pieces of candidate structure information and computes energies thereof. Based on the obtained energies, the analyst prepares a large number of pieces of new candidate structure information and computes energies. These processes are repeated until the energies reach a value less than or equal to an allowable value to identify the crystal structure.

That is, the identification of a crystal structure by the Rietveld method is considered to be an optimization technique using spectra, and the identification of a crystal structure by the density functional theory is considered to be an optimization technique using energy.

The identification of a crystal structure by the Rietveld method generally requires input of candidate structure information regarding a candidate structure very close to a correct crystal structure, and an unknown crystal structure is difficult to identify. The identification of a crystal structure by the density functional theory has an issue that an obtained structure is often different from a structure actually obtained by an experiment.

In contrast, the optimization technique according to Embodiment 2 identifies a crystal structure by an optimization algorithm that uses both spectra and energy. This eliminates the need to input candidate structure information regarding a candidate structure close to a correct crystal structure, and makes it possible to search for a structure similar to an experimentally obtained spectrum, that is, a structure close to an accurate crystal structure.

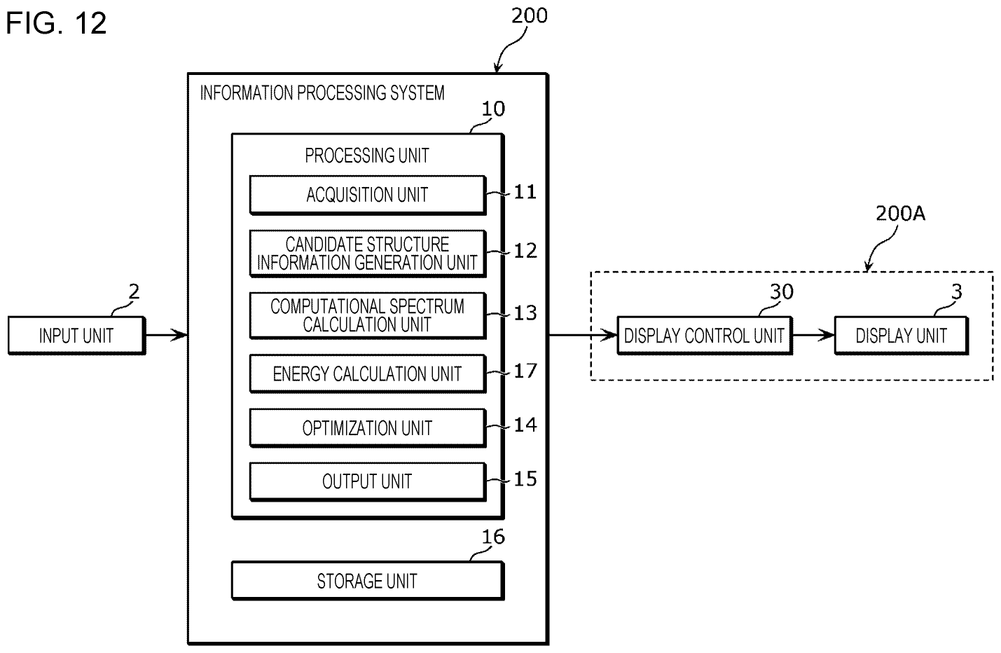

Hereinafter, the information processing system 200 (information processing method or program) according to Embodiment 2 will be described in detail with reference to the drawings. FIG. 12 is a block diagram illustrating an overall configuration including the information processing system 200 according to Embodiment 2. As illustrated in FIG. 12, the information processing system 200 according to Embodiment 2 is different from the information processing system 100 according to Embodiment 1 in that the processing unit 10 further includes an energy calculation unit 17 and the optimization unit 14 further refers to an energy calculated by the energy calculation unit 17. In the following, a description of a configuration common to the information processing system 100 according to Embodiment 1 will be omitted.

The energy calculation unit 17 calculates an energy for each of the candidate structures corresponding to the pieces of candidate structure information generated by the candidate structure information generation unit 12. The energy calculation unit 17 is a subject that executes a step of acquiring energy information indicating an energy calculated for each of the candidate structures in the information processing method according to the present disclosure. Details of the processing executed by the energy calculation unit 17 will be described below.

In Embodiment 2, the optimization unit 14 performs structural optimization on at least one candidate structure among the candidate structures by using the experimental spectrum information acquired by the acquisition unit 11, the computational spectrum information calculated by the computational spectrum calculation unit 13, and the energy information calculated by the energy calculation unit 17. That is, in Embodiment 2, the structural optimization is performed using the experimental spectrum information, the computational spectrum information, and the energy information. In Embodiment 2, furthermore, the structural optimization is performed by a gradient descent method that uses a score obtained by combining the similarity between the experimental spectrum and the computational spectrum with energy. Details of the structural optimization will be described below.

### Operation

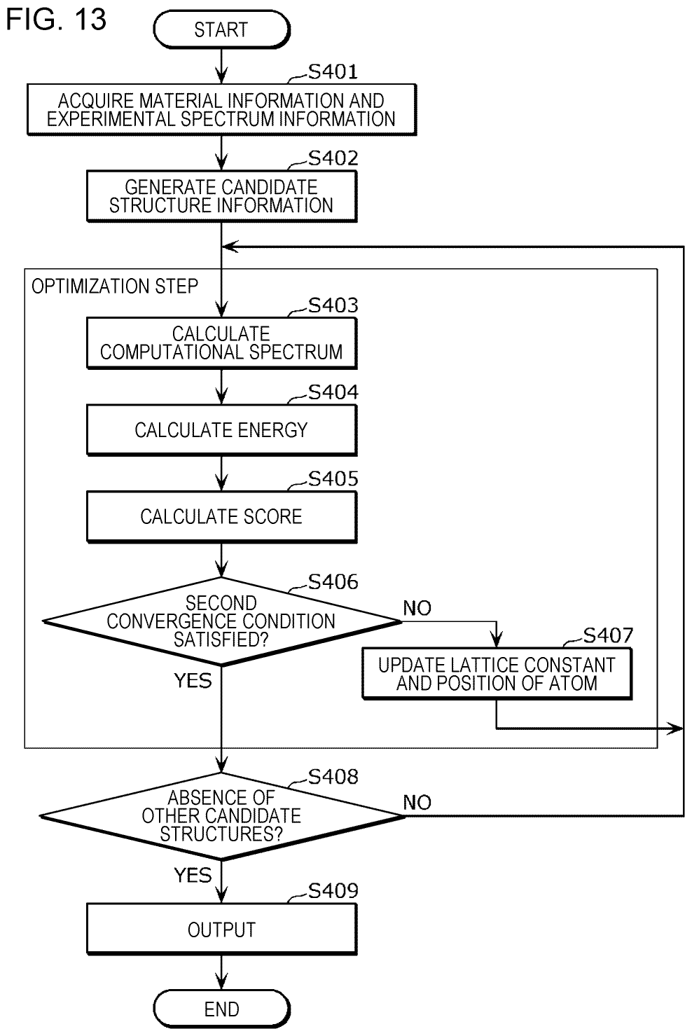

Hereinafter, the operation of the information processing system 200 (that is, an information processing method) according to Embodiment 2 will be described. FIG. 13 is a flowchart illustrating an overview of the operation of the information processing system 200 according to Embodiment 2.

### Step S401

The acquisition unit 11 acquires material information and experimental spectrum information. Step S401 is the same processing as step S101 (see FIG. 2).

### Step S402

The candidate structure information generation unit 12 generates pieces of candidate structure information regarding candidate structures, based on the material information acquired by the acquisition unit 11. Step S402 is the same processing as step S102 (see FIG. 2).

Subsequent steps S403 to S407 correspond to an optimization step. In the information processing system 200 (information processing method) according to Embodiment 2, the structural optimization in the optimization step is performed by a gradient descent method that uses a score obtained by combining the similarity between the experimental spectrum and the computational spectrum with energy.

### Step S403

The computational spectrum calculation unit 13 calculates a computational spectrum for one candidate structure (or structurally optimized structure) among the candidate structures corresponding to the pieces of candidate structure information generated by the candidate structure information generation unit 12. Step S403 is the same processing as step S103 (see FIG. 2).

### Step S404

The energy calculation unit 17 calculates an energy for the candidate structure (or structurally optimized structure) that is a calculation target in the computational spectrum calculation unit 13. The energy to be calculated in step S404 (energy calculation step) is a quantity by which the order is given to a structure group having the composition ratio of the material to be searched for, and is, for example, a quantity obtained by calculating the cohesive energy or the formation energy in atomic units.

In Embodiment 2, the energy calculation unit 17 calculates the energy by using an interatomic potential. Here, the interatomic potential refers to a potential group that describes interactions between atoms. As the interatomic potential, for example, Lennard-Jones, Buckingham, Born-Mayer-Huggins, Stillinger-Weber, Tersoff, Bond-Valence-Site-Energy, or the like can be used. It is also possible to use machine learning or deep learning to approximate an interatomic potential obtained by, for example, the density functional theory, and the result can also be used as an interatomic potential.

This technique enables high-speed computation of energy, and thus facilitates efficient identification of a crystal structure. In general, energy computation based on the interatomic potential can be performed at an overwhelmingly higher speed than energy computation based on the density functional theory. In terms of the accuracy of energy computation, however, the technique using the interatomic potential is inferior to the density functional theory. In the information processing method according to the present disclosure, since the structural optimization is performed using both spectra and energy, the low accuracy of energy calculation by the technique using the interatomic potential does not cause a problem.

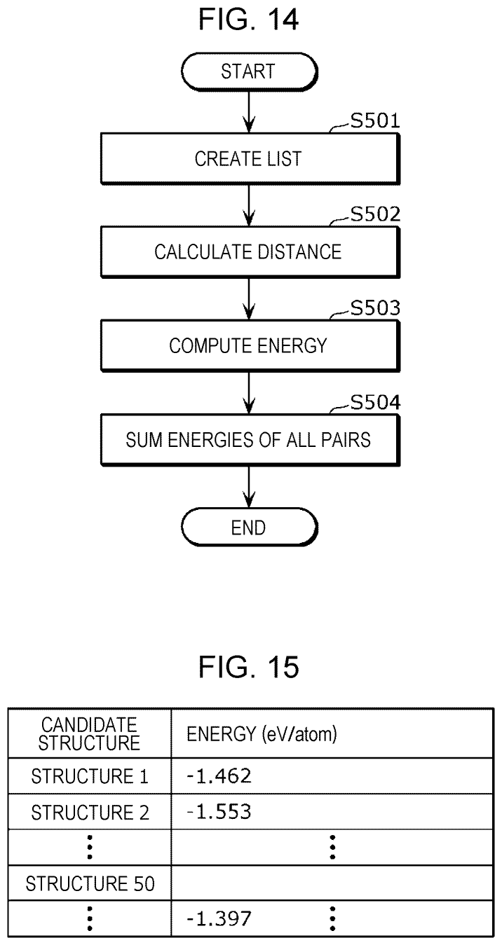

Hereinafter, a procedure for calculating energy using the interatomic potential will be described. FIG. 14 is a flowchart illustrating an example of energy calculation.

### Step S501

The energy calculation unit 17 creates a list of pairs of two atoms for all the atoms included in the candidate structure (or structurally optimized structure). For example, it is assumed that the candidate structure includes a total of eight atoms, namely, “p1”, . . . , and “p8”. In this case, the energy calculation unit 17 creates a list of 28 (=₈C₂) pairs in total, such as “p1-p2” and “p1-p3”.

### Step S502

Next, the energy calculation unit 17 calculates a distance between two atoms for each of all the pairs included in the created list.

### Step S503

Next, the energy calculation unit 17 calculates an energy acting between two atoms for each of all the pairs included in the created list, based on the calculated distance between the two atoms and the atomic number of each of the two atoms.

### Step S504

Then the energy calculation unit 17 sums the energies of all the pairs included in the created list to calculate the energy of the candidate structure (or structurally optimized structure). Here, the energies between all the atoms may be simply totaled, or may be summed with weights for the respective atoms. For example, in a case where only a specific pair of two atoms is to be optimized, it is possible to preferentially optimize the pair of atoms by utilizing such weighting.

FIG. 15 is a diagram illustrating an example of a list of energies of candidate structures. The example illustrated in FIG. 15 is a list of energies of candidate structures when the composition formula of the material to be searched for is “Ca₄Ti₄O₁₂”. A table illustrated in FIG. 15 includes a column indicating the number of a candidate structure, and a column indicating the energy (in eV/atom) of the candidate structure calculated by the energy calculation unit 17.

### Step S405

Referring back to FIG. 13, the optimization unit 14 calculates the similarity between the experimental spectrum acquired by the acquisition unit 11 and the computational spectrum calculated by the computational spectrum calculation unit 13. Then, the optimization unit 14 calculates a weighted average of the calculated similarity and the energy calculated by the energy calculation unit 17 to calculate a score.

### Step S406

Next, the optimization unit 14 determines whether the calculated score satisfies a second convergence condition. The second convergence condition is a predetermined condition based on the score. For example, the second convergence condition is a condition in which the calculated score is greater than or equal to a third threshold in a case where the structural optimization has not been executed even one time. On the other hand, in a case where the structural optimization has been executed one or more times, the second convergence condition is at least one of a second score being greater than or equal to the third threshold and the difference between the second score and a first score being less than or equal to a fourth threshold smaller than the third threshold. The second convergence condition in a case where the structural optimization has been executed one or more times may be “the second score being greater than or equal to a third threshold”, “the difference between the second score and the first score being less than or equal to a fourth threshold smaller than the third threshold”, or “the second score being greater than or equal to the third threshold and the difference between the second score and the first score being less than or equal to the fourth threshold”.

The first score is a score of the candidate structure before the structural optimization is performed. The second score is a score of a structure obtained by the structural optimization. In a case where the structural optimization has been executed multiple times, the second score is a score for the latest structurally optimized structure, and the first score is a score for the structure structurally optimized immediately before the latest structural optimization has been executed.

If the score satisfies the second convergence condition (step S406: Yes), then, the optimization unit 14 executes step S408. On the other hand, if the score does not satisfy the second convergence condition (step S406: No), then, the optimization unit 14 executes step S407. That is, when the score of the candidate structure or a structure obtained by structurally optimizing the candidate structure is greater than or equal to the third threshold, the optimization step ends. When the difference between the second score and the first score is less than or equal to the fourth threshold (in other words, the gradient is less than or equal to the threshold) for the candidate structure or a structure obtained by structurally optimizing the candidate structure, the optimization step ends.

In this way, the structural optimization is executed one or more times on the target candidate structure until the second convergence condition is satisfied. When the score of the candidate structure satisfies the second convergence condition, in some cases, the structural optimization is not executed even one time.

### Step S407

Next, the optimization unit 14 updates a lattice constant and the position of an atom of the candidate structure (or structurally optimized structure). First, the optimization unit 14 calculates a gradient with respect to the score to change the candidate structure (or structurally optimized structure) so that the score improves. The gradient can be calculated by partially differentiating the score with respect to each component of the coordinates of an atom and a lattice constant. Then, the optimization unit 14 applies an optimization algorithm similar to that of Embodiment 1 using the calculated gradient to update a lattice constant and the coordinates of an atom of the candidate structure (or structurally optimized structure). That is, the structural optimization is executed along the gradient direction (that is, the direction in which the score improves) so that the gradient becomes less than or equal to the threshold. Then, the information processing system 200 (information processing method) returns to step S403, and executes the optimization step again.

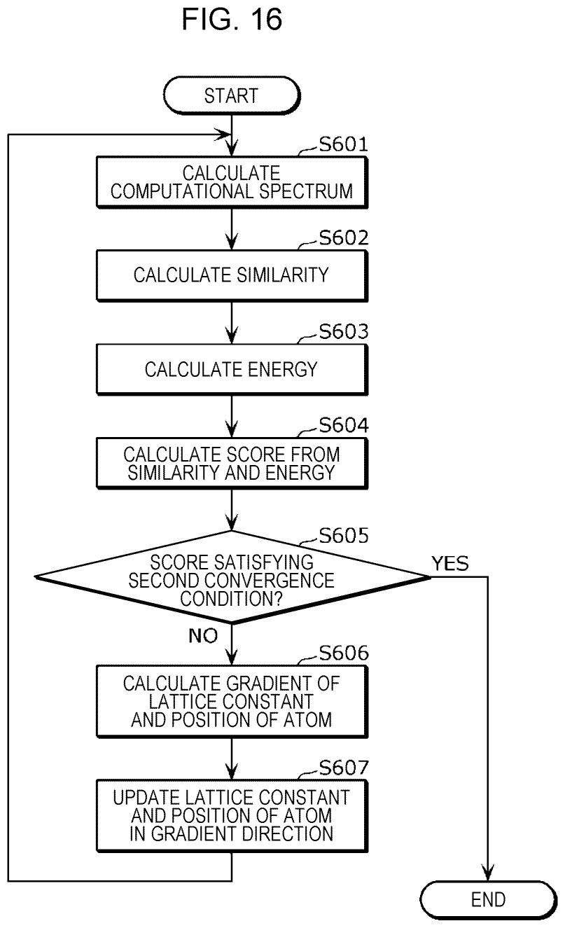

The above-described optimization step is illustrated again in FIG. 16. FIG. 16 is a flowchart illustrating an example of the optimization step performed by the information processing system 200 according to Embodiment 2.

### Step S601

First, the computational spectrum calculation unit 13 calculates a computational spectrum for one candidate structure (or structurally optimized structure) among the candidate structures corresponding to the pieces of candidate structure information generated by the candidate structure information generation unit 12. Step S601 is the same processing as step S403 (see FIG. 13).

### Step S602

Next, the optimization unit 14 calculates the similarity between the experimental spectrum acquired by the acquisition unit 11 and the computational spectrum calculated by the computational spectrum calculation unit 13. Step S602 is the same processing as part of step S405 (see FIG. 13).

### Step S603

Next, the energy calculation unit 17 calculates an energy for the candidate structure (or structurally optimized structure) that is a calculation target in the computational spectrum calculation unit 13. Step S603 is the same processing as step S404 (see FIG. 13). Step S602 and step S603 may be executed in reverse order.

### Step S604

Next, the optimization unit 14 calculates a weighted average of the calculated similarity and the energy calculated by the energy calculation unit 17 to calculate a score. Step S604 is the same processing as step S405 (see FIG. 13).

### Step S605

Next, the optimization unit 14 determines whether the calculated score satisfies a second convergence condition. Step S605 is the same processing as step S406 (see FIG. 13). If the score satisfies the second convergence condition (step S605: Yes), the optimization step ends. On the other hand, if the score does not satisfy the second convergence condition (step S605: No), then, the optimization unit 14 executes step S606.

### Step S606

Next, the optimization unit 14 calculates a gradient of a lattice constant and the position of an atom of the candidate structure (or structurally optimized structure). Step S606 is the same processing as part of step S407 (see FIG. 13).

### Step S607

Next, the optimization unit 14 changes a lattice constant and the position of an atom of the candidate structure (or structurally optimized structure) along the calculated gradient direction to update the lattice constant and the position of the atom. Step S607 is the same processing as part of step S407 (see FIG. 13). Then, the information processing system 200 (information processing method) returns to step S601, and executes the optimization step again.

### Step S408

Returning to FIG. 13, the optimization unit 14 determines whether any other candidate structure is present. If any other candidate structure is present (step S408: No), the process returns to step S403, and the optimization step is executed on any other candidate structure. On the other hand, if no other candidate structure is present (step S408: Yes), then, the information processing system 200 (information processing method) executes step S409.

### Step S409

The output unit 15 outputs the structure information generated by the optimization unit 14. In step S409 (output step), one or more locally stable structures obtained in the optimization step are output as structure information. The structure information includes information regarding lattice constants and the positions of atoms of one or more structures generated by the optimization unit 14. The output format of the structure information is not particularly limited, and may be, for example, a format in which parameters are simply listed or a standardized format such as a Crystallographic Information File (CIF). Here, the output unit 15 causes the display unit 3 to display the second image representing the structure information generated by the optimization unit 14 to output the structure information.

The output unit 15 may output one or more pieces of structure information determined in the optimization step. The output unit 15 may cause the display unit 3 to display the one or more pieces of structure information. Each of the one or more pieces of structure information may correspond to one piece of material information acquired in step S401, that is, one composition formula. In step S409 (output step), the one or more pieces of structure information may be output. Each of the one or more pieces of structure information may include information regarding lattice constants and the positions of atoms that determine each of one or more candidate structures that are one or more candidates of the crystal structure corresponding to the composition formula.

As described above, in Embodiment 2, the candidate structure is optimized so that the candidate structure is thermodynamically stable and the experimental spectrum and the computational spectrum coincide with each other. Thus, more realistic identification of a crystal structure can be achieved. In Embodiment 2, a gradient is calculated for a score calculated from a weighted average of the similarity and the energy. Therefore, adjusting the respective coefficients of the similarity and the energy to calculate a score makes it possible to optimize the candidate structure while balancing the thermodynamic validity and the spectral consistency. Hereinafter, an effect of using the gradient for the score obtained by adding energy to the similarity of a spectrum will be described.

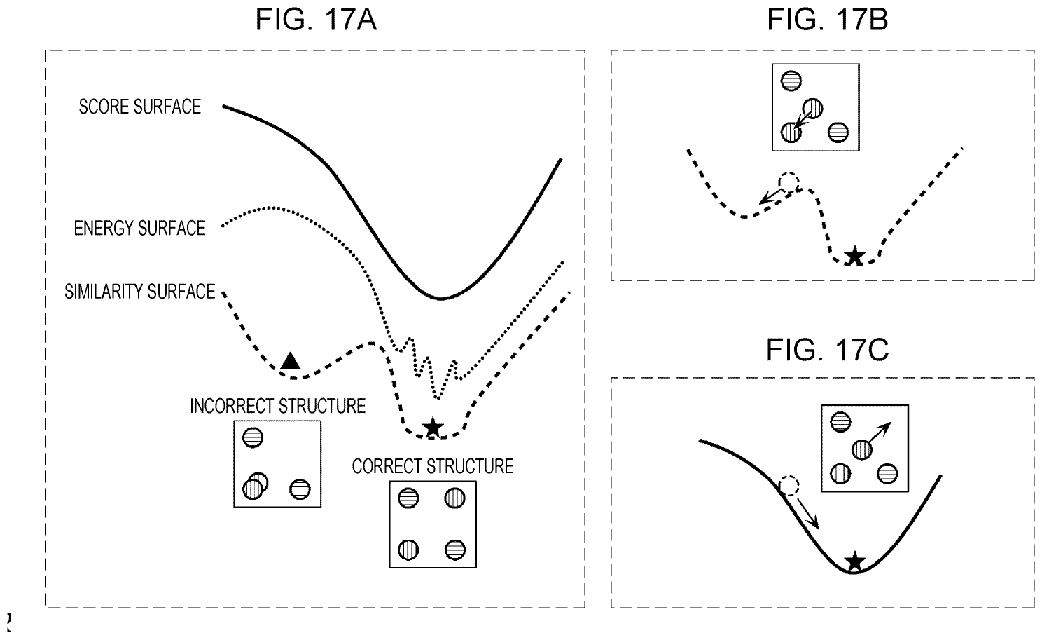

FIGS. 17A, 17B, and 17C are explanatory diagrams of structural optimization by the information processing system 200 according to Embodiment 2. In FIG. 17A, a solid line represents a score surface, a dotted line represents an energy surface, and a broken line represents a similarity surface. In an example illustrated in FIG. 17A, two valleys are present in the similarity surface. When an optimized structure obtained after the structural optimization is executed one or more times is located in one of the valleys, the optimized structure is a locally stable structure with a relatively high similarity, that is, a correct structure. When the optimized structure is located in the other valley, however, the optimized structure is an incorrect structure although the structure has relatively low similarity and appears to be locally stable at first glance.

An experimental spectrum of an X-ray diffraction pattern is generally obtained from a crystal structure with a high symmetry. Accordingly, a crystal structure having a computational spectrum exhibiting a high similarity to the experimental spectrum has an aligned atomic arrangement like the correct structure as illustrated in FIG. 17A. Thus, through an optimization operation using a gradient of the similarity, the atomic group of the random initial arrangement is optimized in the direction in which the atomic arrangement is aligned. At this time, in the similarity surface, as illustrated in FIG. 17B, the structural optimization is not performed along a gradient toward the correct structure, but the structural optimization is performed along a gradient toward the incorrect structure, and atoms may overlap at the same coordinates. The arrangement of such atoms is very unstable in terms of energy, and could not occur in nature.

In the score surface, by contrast, as illustrated in FIG. 17C, the structural optimization is performed along a gradient toward the correct structure. That is, in the structural optimization using the gradient with respect to the score obtained by adding the energy and the similarity, the arrangement in which atoms overlap is disadvantageous in terms of the score, and thus the gradient direction is not a direction toward the incorrect structure but a direction toward the correct structure. In Embodiment 2, accordingly, it is possible to avoid the arrangement of atoms that have a high similarity but are unstable in terms of energy, and it is possible to perform structural optimization to achieve a crystal structure having a low energy and a high similarity, that is, a structure close to an unknown crystal structure of the material to be searched for.

In Embodiment 2, the optimization step using only the similarity may be additionally performed on a structure on which the optimization step using the score is performed. Therefore, the similarity can further be increased in the finally obtained crystal structure.

### User Interface

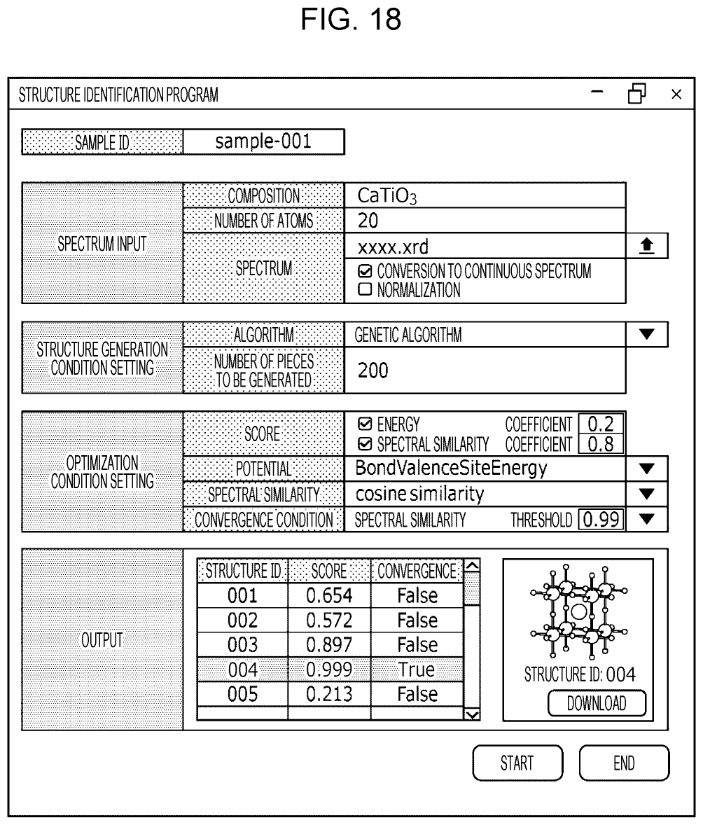

Hereinafter, a user interface of the information processing system 200 according to Embodiment 2 will be described with reference to the drawings. FIG. 18 is a diagram illustrating an image displayed on the display unit 3 in the information processing system 200 according to Embodiment 2. In FIG. 18, a “spectrum input” area corresponds to the first image. The first image may correspond to an area including the “spectrum input” area, a “structure generation condition setting” area, and an “optimization condition setting” area. In FIG. 18, an “output” area corresponds to the second image.

The “spectrum input” area displays a text box for inputting a composition formula of a material, a text box for inputting the number of atoms included in a crystal structure, a button for selecting and uploading data of an experimentally obtained X-ray diffraction pattern, a check box for selecting whether to execute conversion processing into a continuous spectrum, and a check box for selecting whether to execute normalization processing.

The “structure generation condition setting” area displays a pull-down menu for selecting a method for generating candidate structure information regarding a candidate structure, and a text box for inputting the number of pieces of candidate structure information regarding candidate structures to be generated.

The “optimization condition setting” area displays a text box for inputting a weighting coefficient of each of the energy and the similarity to calculate a score, a pull-down menu for selecting an interatomic potential, a pull-down menu for selecting an index for evaluating the similarity, a pull-down menu for selecting a convergence condition, and a text box for inputting a threshold of the convergence condition.

The “output” area displays a list of one or more structures that are structurally optimized, and an image of a structure selected by the user. The image is a candidate image of a crystal structure corresponding to the composition formula CaTiO₃ identified by the structure ID=004 in FIG. 18. The list includes, in order from the left, a column indicating an identification number (ID) of each structure, a column indicating the score of the structure, and a column indicating whether the structure satisfies the convergence condition. A case where the structure does not satisfy the convergence condition (that is, “False”) corresponds to, for example, a case where the convergence condition is not satisfied even when the structural optimization is performed a predetermined number of times or more.

When the user selects a download button included in the “output” area in FIG. 18, the processing unit 10 receives a signal indicating that the download button has been selected. When the processing unit 10 receives the signal of the selection, the processing unit 10 sends an instruction to the display control unit 30 to cause the display unit 3 to display lattice constants and sites (that is, atomic positions) of the crystal structure corresponding to the composition formula CaTiO₃ identified by the structure ID=004. When the display control unit 30 receives the instruction, the display control unit 30 causes the display unit 3 to display the lattice constants and the sites (that is, atomic positions) of the crystal structure corresponding to the composition formula CaTiO₃ identified by the structure ID=004. The lattice constants and the sites of the crystal structure may be expressed in the format illustrated in FIG. 5B. That is, the display unit 3 may display the respective values of the lattice constants a, b, c, α, β, and γ of the crystal structure corresponding to the composition formula CaTiO₃ identified by the structure ID=004, the three-dimensional coordinates of one Ca atom, the three-dimensional coordinates of one Ti atom, and the three-dimensional coordinates of each of three O atoms.

The list displayed in the “output” area may include pieces of information ᵢ, where i is a natural number greater than or equal to 2 and less than or equal to n, and n is the number of candidate structures. The information ᵢ=[the structure IDᵢ, the score ᵢ, information as to whether the candidate structure identified by the structure ID; satisfies or does not satisfy the convergence condition, the lattice constants of the candidate structure identified by the structure IDᵢ, and the respective positions of the atoms included in the candidate structure identified by the structure IDᵢ]. For example, the structure ID₄ included in the information ₄ is 004, and the score ₄ included in the information ₄ is 0.999.

When the candidate structure identified by the structure IDᵢ does not satisfy the convergence condition (that is, when “False” is indicated in the “convergence” column of the list included in the “output” area in FIG. 18), the list does not include the lattice constants of the candidate structure identified by the structure ID; and does not include the respective positions of the atoms included in the candidate structure identified by the structure IDᵢ.

The “output” area in FIG. 18 may include information illustrated in FIG. 27. FIG. 27 is a diagram illustrating an example of information included in the “output” area in FIG. 18. FIG. 27 illustrates lattice constants of a crystal structure corresponding to a structure ID and atomic positions included in the crystal structure. The information illustrated in FIG. 27 includes one or more structure IDs. Each of one or more candidate structures corresponding one-to-one to the one or more structure IDs satisfies the convergence condition. The description of the lattice constants and the description of the positions of the atoms in FIG. 27 can be understood by referring to the description of the lattice constants and the description of the positions of the atoms in FIG. 26.

The user inputs desired parameters in the “spectrum input” area, the “structure generation condition setting” area, and the “optimization condition setting” area, and then selects a “start” icon. Accordingly, the information processing system 200 executes the series of processes, and the processing result is displayed in the “output” area.

### EXAMPLES

Hereinafter, an example (Example 1) of the information processing system 100 according to Embodiment 1 and an example (Example 2) of the information processing system 200 according to Embodiment 2 will be described in comparison with an example (Comparative Example 1) of an information processing system according to Comparative Example 1 and an example (Comparative Example 2) of an information processing system according to Comparative Example 2.

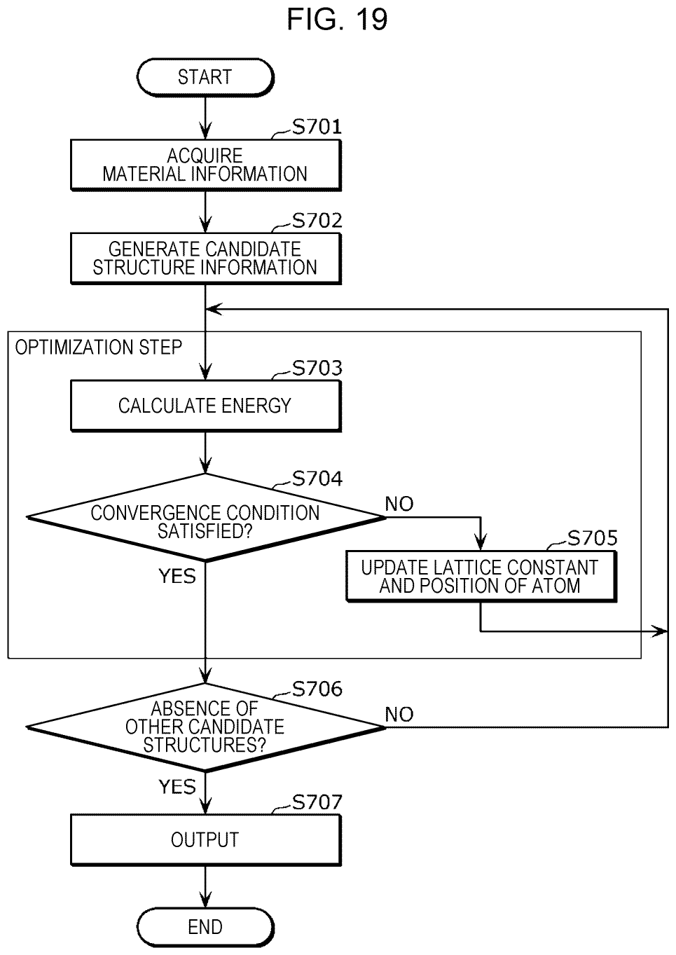

FIG. 19 is a flowchart illustrating an overview of the operation of the information processing system according to Comparative Example 1. The information processing system according to Comparative Example 1 is different from the information processing system 100 according to Embodiment 1 in that an energy is calculated instead of a computational spectrum and the structural optimization is performed so that the energy satisfies a convergence condition (that is, the energy is less than or equal to a threshold).

In the flowchart illustrated in FIG. 19, step S701 is the same processing as step S101 (see FIG. 2) except that the experimental spectrum information is not acquired. Further, step S702 is the same processing as step S102 (see FIG. 2). Further, the optimization step (steps S703 to S705) is different from the optimization step (steps S103 to S106 (see FIG. 2)) of the information processing system 100 according to Embodiment 1 in that the optimization step is performed by a gradient descent method that uses energy instead of the similarity between the experimental spectrum and the computational spectrum. Further, step S706 is the same processing as step S107 (see FIG. 2). Further, step S707 is the same processing as step S108 (see FIG. 2).

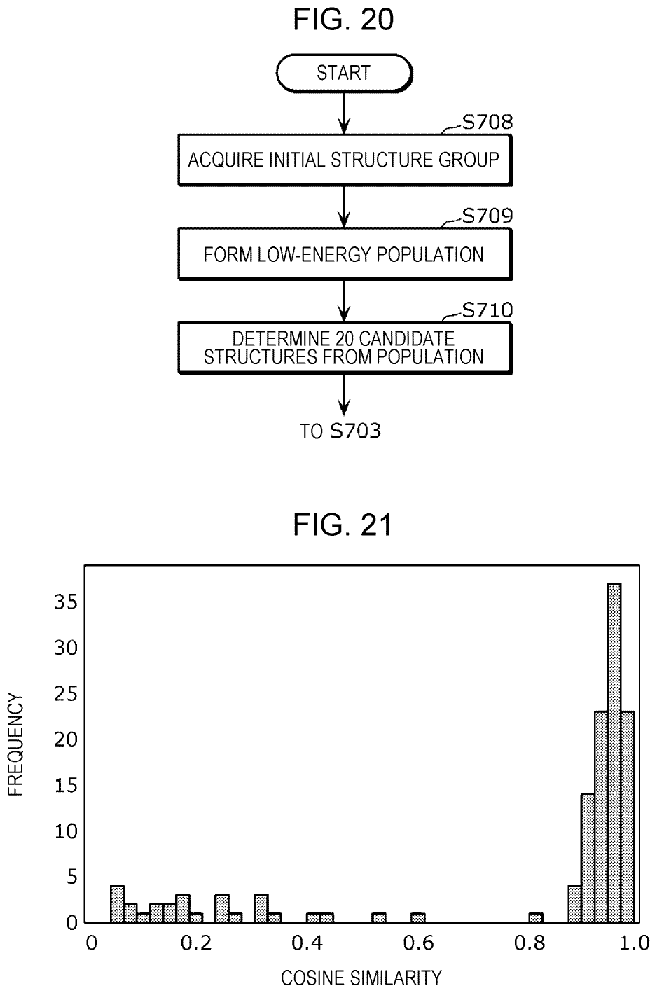

FIG. 20 is a flowchart illustrating an overview of the operation of the information processing system according to Comparative Example 2. The information processing system according to Comparative Example 2 determines a candidate structure from an initial structure group by using a genetic algorithm, the initial structure group including structurally optimized structures obtained by the information processing system according to Comparative Example 1. The processing after a candidate structure is determined is the same as that of the information processing system according to Comparative Example 1.

In the flowchart illustrated in FIG. 20, step S708 is a process of acquiring initial structure groups described above. Step S709 is a process of forming a population having a relatively low energy from the acquired initial structure groups. Step S710 is a process of determining a predetermined number of (here, 20) candidate structures from the formed population. In step S710, an operation such as exchange of atoms, displacement or inversion of the positions of atoms, or displacement of lattice constants is performed. Steps S709 and S710 may be repeatedly executed multiple times instead of one time.

### Example 1

First, the example (Example 1) of the information processing system 100 according to Embodiment 1 will be described.

### Acquisition of Experimental Spectrum

From an X-ray diffraction pattern of orthorhombic CaTiO₃ having the same composition ratio as Ca₄Ti₄O₁₂, a group of peaks having an intensity greater than or equal to 1/100 of the peak having the highest intensity was extracted. A Gaussian function (standard deviation: 0.5) was applied to the positions of these peaks to obtain a continuous pattern (continuous spectrum) of 0 to 90°. This was normalized by the highest peak intensity to obtain an experimental spectrum.

### Determination of Candidate Structure

For each of the space groups numbered 2 to 230, one candidate structure having a composition of Ca₄Ti₄O₁₂ was determined. At this time, the initial atomic arrangement and lattice constants were randomly generated from parameters satisfying the symmetry of the space groups. In addition, a space group that could not be reproduced by a composition of Ca₄Ti₄O₁₂ was excluded. Through this operation, a total of 129 candidate structures were determined (that is, 129 pieces of candidate structure information were generated).

### Calculation of Computational Spectrum

X-ray diffraction patterns were calculated for the candidate structures (or structurally optimized structures), and a Gaussian function (standard deviation: 0.5) was applied to obtain continuous patterns (continuous spectra) of 0 to 90°. This was normalized by the highest peak intensity to obtain computational spectra.

### Structural Optimization

The gradient descent method for spectral similarity was used to update the position of an atom and a lattice constant. The update of the position of the atom and the lattice constant was performed by the Adam algorithm. The structural optimization was performed until the standard deviations of the similarities in the most recent 10 steps became 1E-06. When the convergence condition (first convergence condition) was not satisfied in 1000 steps, the computation was terminated, and a structure obtained in the final step was set as the finally structurally optimized structure.

### Example 2

Next, the example (Example 2) of the information processing system 200 according to Embodiment 2 will be described.

Processing similar to that in Example 1 was performed to acquire an experimental spectrum, determine candidate structures (that is, generate pieces of candidate structure information), and calculate computational spectra.

### Energy Calculation

The energies of the candidate structures (or structurally optimized structures) were calculated using Bond-Valence-Site-Energy. A screening factor indicating the contribution of the Coulomb potential was set to 0.7.

### Structural Optimization

The gradient descent method for a score obtained by calculating a weighted average of the energies and the spectral similarity was used to update the position of an atom and a lattice constant. The update of the position of the atom and the lattice constant was performed by the Adam algorithm. The structural optimization was performed until the standard deviations of the scores in the most recent 10 steps became 1E-06. When the convergence condition (second convergence condition) was not satisfied in 1000 steps, the computation was terminated, and a structure obtained in the final step was set as the finally structurally optimized structure.

### Comparative Example 1

Next, the example (Comparative Example 1) of the information processing system according to Comparative Example 1 will be described.

### Determination of Candidate Structure

Through processing similar to that in Examples 1 and 2, 129 candidate structures were determined (that is, 129 pieces of candidate structure information were generated).

### Energy Calculation

The energies of the candidate structures (or structurally optimized structures) were calculated by processing similar to that in Example 2.

### Structural Optimization

The gradient descent method for energy was used to update the position of an atom and a lattice constant. The update of the position of the atom and the lattice constant was performed by the Adam algorithm. The structural optimization was performed until the standard deviations of the energies in the most recent 10 steps became less than or equal to 1E-06. When the convergence condition was not satisfied in 1000 steps, the computation was terminated, and a structure obtained in the final step was set as the finally structurally optimized structure.

### Comparative Example 2

Next, the example (Comparative Example 2) of the information processing system according to Comparative Example 2 will be described. In Comparative Example 2, for an initial structure group including structures structurally optimized in Comparative Example 1, 200 candidate structures were determined by using a genetic algorithm (that is, 200 pieces of candidate structure information were generated). The processing in Comparative Example 2 is the same as that in Comparative Example 1 except for the determination of candidate structures (that is, the generation of pieces of candidate structure information). Determination of Candidate Structure

The candidate structures were determined by using a genetic algorithm (that is, the pieces of candidate structure information were generated by using a genetic algorithm). The structure group structurally optimized in Comparative Example 1 was used as an initial structure group. From a population having a low energy after the structural optimization within the initial structure group, 20 child structures were generated. This process was repeated 10 times to determine a total of 200 candidate structures (that is, generate 200 pieces of candidate structure information).

### Results

FIG. 21 is a histogram illustrating similarities between an experimental spectrum and computational spectra of a structure group structurally optimized in Example 1. As illustrated in FIG. 21, the computational spectra of 79% of the structures in the structure group have a cosine similarity of 0.8 or more with the experimental spectrum, and convergence to a structure similar to the actual crystal structure is found to have been obtained. In addition, the cosine similarity reached up to 0.992, and a tilted skeleton specific to the orthorhombic CaTiO₃ was reproduced.

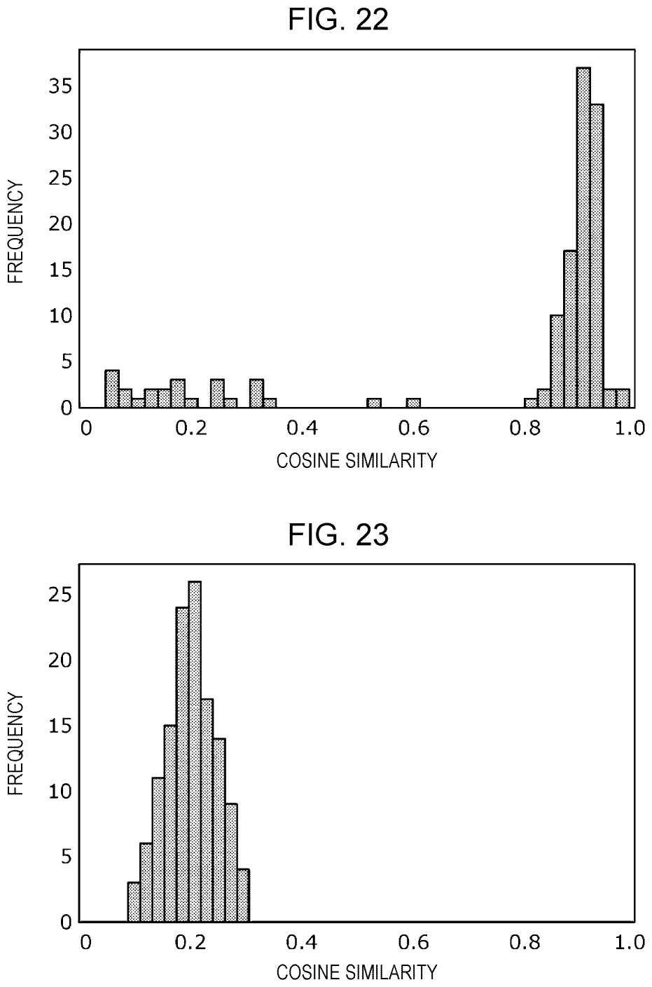

FIG. 22 is a histogram illustrating similarities between an experimental spectrum and computational spectra of a structure group structurally optimized in Example 2. As illustrated in FIG. 22, the computational spectra of 81% of the structures in the structure group have a cosine similarity of 0.8 or more with the experimental spectrum, and convergence to a structure similar to the actual crystal structure is found to have been obtained. In addition, the cosine similarity reached up to 0.998, and a tilted skeleton specific to the orthorhombic CaTiO₃ was reproduced. As described above, it is found that the information processing method according to the present disclosure enables efficient identification of a crystal structure.

FIG. 23 is a histogram illustrating similarities between an experimental spectrum and computational spectra of a structure group structurally optimized in Comparative Example 1. As illustrated in FIG. 23, a cosine similarity up to 0.31 is achieved between the computational spectra and the experimental spectrum, and convergence to a structure different from the actual crystal structure is found to have been obtained.

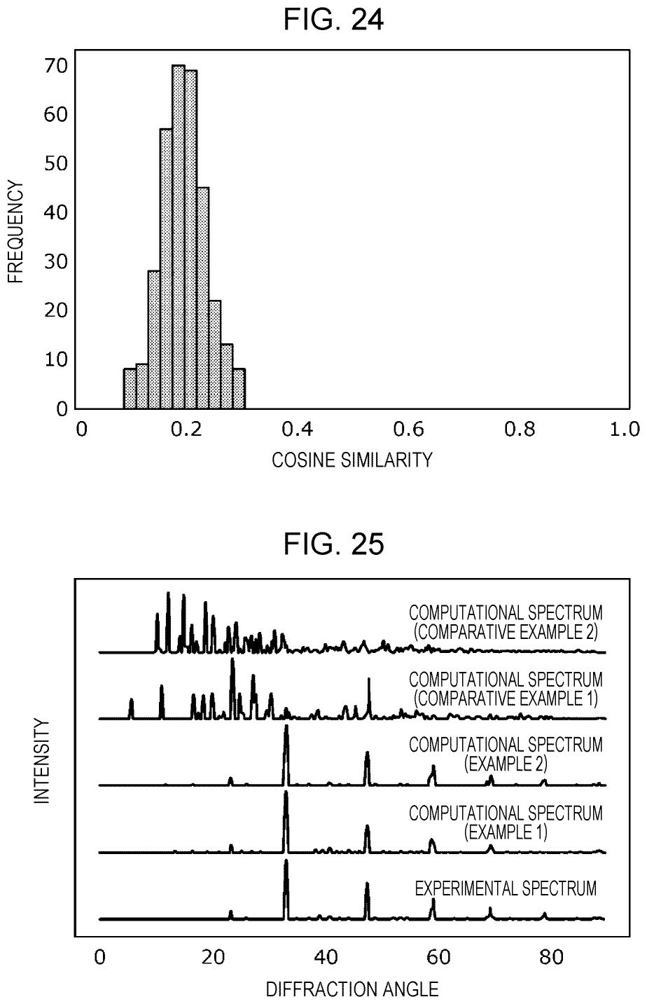

FIG. 24 is a histogram illustrating similarities between an experimental spectrum and computational spectra of a structure group structurally optimized in Comparative Example 2. As illustrated in FIG. 24, a cosine similarity up to 0.33 is achieved between the computational spectra and the experimental spectrum, and convergence to a structure different from the actual crystal structure is found to have been obtained.

FIG. 25 is a diagram illustrating a comparison between an experimental spectrum and computational spectra of structures having the highest similarity in Examples 1 and 2 and Comparative Examples 1 and 2. As illustrated in FIG. 25, it is found that the computational spectra in Examples 1 and 2 have almost completely the same spectrum shape as the experimental spectrum. By contrast, it is found that the computational spectra in Comparative Examples 1 and 2 have completely different shapes from the experimental spectrum. In FIG. 25, the vertical axis represents intensity, and the horizontal axis represents 2θ (“2θ” is a diffraction angle). The unit of 2θ is an angle.

From the above results, as in Comparative Examples 1 and 2, the identification of a crystal structure without using a computational spectrum makes it difficult to obtain a structure close to an actual crystal structure even if a large number of candidate structures are optimized. As in Examples 1 and 2, by contrast, the identification of a crystal structure using a computational spectrum makes it possible to efficiently identify an actual crystal structure.

### Modifications

While an information processing system (information processing method) according to one or more aspects of the present disclosure has been described above with reference to embodiments, the present disclosure is not limited to these embodiments. Various modifications conceivable to a person skilled in the art may be made to the embodiments described above without departing from the spirit of the present disclosure, and such modifications may also be encompassed in the present disclosure. In addition, embodiments resulting from combinations of components in different embodiments may also be included in the present disclosure.

For example, in the embodiments described above, the information processing systems 100 and 200 cause the display unit 3 to display the first image or the second image, but the present disclosure is not limited thereto. For example, the information processing systems 100 and 200 do not cause the display unit 3 to display the first image or the second image, but may output information included in these images.

In the embodiments described above, furthermore, the information processing systems 100 and 200 include the processing unit 10 and the storage unit 16, but the present disclosure is not limited thereto. For example, as indicated by “100A” in FIG. 1, the information processing system according to Embodiment 1 may include the display control unit 30 and the display unit 3. For example, as indicated by “200A” in FIG. 12, the information processing system according to Embodiment 2 may include the display control unit 30 and the display unit 3.

In the embodiments described above, each component may be configured by dedicated hardware or may be implemented by executing a software program suitable for each component. Each component may be implemented by a program execution unit such as a central processing unit (CPU) or a processor reading and executing a software program recorded on a recording medium such as a hard disk or a semiconductor memory.

The present disclosure also includes the following cases.

(1) The at least one apparatus described above is specifically a computer system including a microprocessor, a read only memory (ROM), a random access memory (RAM), a hard disk unit, a display unit, a keyboard, a mouse, and so on. The RAM or the hard disk unit stores a computer program. The microprocessor operates in accordance with the computer program, and accordingly the at least one apparatus described above achieves a function thereof. The computer program is configured as a combination of instruction codes indicating instructions given to a computer to achieve a predetermined function.

(2) Some or all of the components of the at least one apparatus described above may include a single system large scale integration (LSI). The system LSI is a super-multifunctional LSI manufactured by integrating constituents on a single chip, and is specifically a computer system including a microprocessor, a ROM, a RAM, and so on. The RAM stores a computer program. The microprocessor operates in accordance with the computer program, and accordingly the system LSI achieves a function thereof.

(3) Some or all of the components of the at least one apparatus described above may include an IC card removably attachable to the apparatus, or a stand-alone module. The IC card or the module is a computer system including a microprocessor, a ROM, a RAM, and so on. The IC card or the module may include the super-multifunctional LSI described above. The microprocessor operates in accordance with the computer program, and accordingly the IC card or the module achieves a function thereof. This IC card or this module may be tamper-resistant.

(4) The present disclosure may provide the methods described above. The present disclosure may provide a computer program for causing a computer to implement these methods, or may provide a digital signal including the computer program.

The present disclosure may provide a computer-readable recording medium on which the computer program or the digital signal is recorded. Examples of such a computer-readable recording medium include a flexible disk, a hard disk, a compact disc (CD)-ROM, a DVD, a DVD-ROM, a DVD-RAM, a Blu-ray (registered trademark) Disc) (BD), and a semiconductor memory. The present disclosure may provide the digital signal recorded on such a recording medium.

In the present disclosure, the computer program or the digital signal may be transmitted via a telecommunication line, a wireless or wired communication line, a network represented by the Internet, data broadcasting, or the like.

The program or the digital signal may be executed by another independent computer system by being recorded on a recording medium and transferred, or by being transferred via a network or the like.

### Other 1

In the present disclosure, at least one of A and B means “A”, “B”, or “A and B”.

### Other 2

A modification of an embodiment of the present disclosure may be as follows.

A method being performed by one or more processors configured to execute instructions stored in one or more memories,
the method including:
acquiring (for example, S101) first spectrum information indicating a first spectrum obtained by measuring a material having a crystal structure;
acquiring (for example, S101) a composition formula for the material;
generating (for example, S102) pieces of first information indicating first candidates of the crystal structure, based on the composition formula;
calculating (for example, S103) second spectrum information corresponding to a second spectrum corresponding to each of the first candidates, based on the pieces of first information, to calculate pieces of second spectrum information indicating second spectra corresponding one-to-one to the pieces of first information;
generating (for example, S104 to S107, S103) one or more pieces of structure information indicating one or more candidate structures that are one or more second candidates of the crystal structure, based on a correlation between each of the pieces of second spectrum information and the first spectrum information; and
outputting (for example, S108) the generated one or more pieces of structure information.

The present disclosure is useful for identifying a crystal structure of an unknown material.

What is claimed is:

1. An information processing method to be executed by a computer, the information processing method comprising:
acquiring experimental spectrum information indicating an experimental spectrum obtained by actually measuring a material to be searched for;
acquiring material information regarding a composition of the material;
generating, based on the material information, pieces of candidate structure information regarding candidate structures that are candidates for a crystal structure of the material, and acquiring computational spectrum information indicating computational spectra each corresponding to a corresponding one of the candidate structures;
generating structure information regarding the crystal structure, based on a correlation between the experimental spectrum information and the computational spectrum information; and
outputting the generated structure information.

2. The information processing method according to claim 1, wherein
the generating of the structure information performs structural optimization on two or more candidate structures among the candidate structures using the experimental spectrum and the computational spectra, and
the structure information indicates a structure obtained by the structural optimization.

3. The information processing method according to claim 2, wherein
the structural optimization is performed using a similarity between the experimental spectrum and the computational spectra, and
the structure information indicates a structure in which the similarity is greater than or equal to a predetermined threshold among two or more structures obtained by the structural optimization.

4. The information processing method according to claim 2, wherein
the structural optimization is performed by changing at least one of a lattice constant of the candidate structures or a position of an atom in the candidate structures, and
the structural optimization is performed such that a second similarity between the experimental spectrum and a computational spectrum of a structure obtained by the structural optimization is higher than a first similarity between the experimental spectrum and a computational spectrum of the candidate structures before the structural optimization is performed.

5. The information processing method according to claim 4, wherein
the structural optimization is executed one or more times on a target candidate structure of the candidate structures until a first convergence condition is satisfied, and
the first convergence condition is at least one of the second similarity being greater than or equal to a first threshold or a difference between the second similarity and the first similarity being less than or equal to a second threshold smaller than the first threshold.

6. The information processing method according to claim 2, wherein
the structural optimization is performed by a gradient descent method that uses a similarity between the experimental spectrum and the computational spectra.

7. The information processing method according to claim 2, further comprising
acquiring energy information indicating an energy calculated for each of the candidate structures, wherein
the structural optimization is performed using the experimental spectrum information, the computational spectrum information, and the energy information.

8. The information processing method according to claim 7, wherein
the structural optimization is performed by a gradient descent method that uses a score obtained by combining a similarity between the experimental spectrum and the computational spectra with the energy.

9. The information processing method according to claim 8, wherein
the structural optimization is executed one or more times on a target candidate structure of the candidate structures until a second convergence condition is satisfied, and
the second convergence condition is a predetermined condition based on the score.

10. The information processing method according to claim 1, wherein
the experimental spectrum and the computational spectra are spectra obtained by an X-ray diffraction method.

11. An information processing method to be executed by a computer, the information processing method comprising:
generating structure information regarding a crystal structure of a material to be searched for, based on a correlation between experimental spectrum information and computational spectrum information, the experimental spectrum information indicating an experimental spectrum obtained by actually measuring the material, the computational spectrum information indicating a computational spectrum calculated for each of candidate structures that are candidates for the crystal structure; and
outputting the generated structure information.

12. An information processing system comprising:
a display controller, wherein
after a first image for receiving an input of material information regarding a composition of a material to be searched for and experimental spectrum information indicating an experimental spectrum obtained by actually measuring the material is displayed on a display, the display controller causes the display to display a second image generated based on the material information and the experimental spectrum information that are input and representing structure information regarding a crystal structure of the material.

13. A non-transitory computer-readable recording medium storing a program for causing a computer to execute processing comprising:
acquiring experimental spectrum information indicating an experimental spectrum obtained by actually measuring a material to be searched for;
acquiring material information regarding a composition of the material;
generating, based on the material information, pieces of candidate structure information regarding candidate structures that are candidates for a crystal structure of the material, and acquiring computational spectrum information indicating computational spectra each corresponding to a corresponding one of the candidate structures;
generating structure information regarding the crystal structure, based on a correlation between the experimental spectrum information and the computational spectrum information; and
outputting the generated structure information.
